(19) United States
(12) Patent Application Publication
(10) Pub. No.: US 2002/0146694 A1
(43) Pub. Date: Oct. 10, 2002

(54) Functionating genomes with cross-species coregulation

(76) Inventors: Stephen H. Friend, Seattle, WA (US); Roland Stoughton, San Diego, CA (US); Matthew J. Marton, Seattle, WA (US); Yudong He, Kirkland, WA (US)

Correspondence Address: PENNIE AND EDMONDS, 1155 AVENUE OF THE AMERICAS, NEW YORK, NY, 100362711

(21) Appl. No.: 09/779,004

(22) Filed: Feb. 7, 2001

(51) Int. Cl. C12Q 1/68; G06F 19/00; G01N 33/48; G01N 33/50

(52) U.S. Cl. 435/6; 702/20

(57) ABSTRACT

The present invention relates to the characterization of genes and their gene products (i.e., proteins). In particular, the invention relates to novel systems and methods for characterizing the cellular function and/or activity of different cellular constituents such as different genes and/or their gene products. The invention also provides novel systems and methods for comparing different cellular constituents (e.g., novel genes and/or their gene products) from different cells, such as genes and/or gene products from cells of different species of organism or, alternatively, from different cells (e.g., of different cell types or from different tissues types) of the same organism. In particular, using the systems and methods of the invention, it is possible to identify different cellular constituents having common cellular functions.

### 1. FIELD OF THE INVENTION

The field of this invention relates to the characterization of genes and their gene products (e.g., proteins). In particular, the invention relates to novel methods and compositions for characterizing the function, and in particular the cellular function, of individual genes and their gene products. The invention also relates to methods and compositions for comparing different genes and gene products, from the same species or from different species, and identifying genes and gene products that have common cellular functions.

### 2. BACKGROUND OF THE INVENTION

Recent and rapid increases in the rate at which DNA sequences are determined, combined with current efforts to sequence the entire human genome and the genomes of other organisms has resulted in the identification of tens of thousands of novel genes that are expressed in many different organisms. Although the nucleotide sequences of these genes have been determined, the biological functions, i.e., molecular, cellular and organismal functions, of many of these genes and/or the gene products (e.g., proteins) they encode remain unknown. Yet knowledge of the cellular function (i.e., the role in a particular cell type) of these novel genes is essential for using the genes, e.g., to identify new molecular targets for medical treatments and interventions, medical diagnostics and genetic engineering (e.g., of plants and livestock), to name a few applications. There has become an urgent need, therefore, to characterize (i.e., determine the cellular function of) a large number of novel genes and/or of their associated gene products. Further, this need will undoubtedly continue to increase as the rate at which novel genes are identified and sequenced continues to accelerate.

Although techniques are already known that may provide insight into the cellular function of novel genes and their gene products, many of these techniques suffer from low throughput rates that are inadequate in view of the current numbers of new genes being sequenced. Other techniques do not have throughput limitations but often provide incomplete information or worse still, useless or inaccurate information. For example, an approach that has become increasingly popular in recent years is to search databases, such as the GenBank database, for genes of known molecular or cellular function that have similar nucleic acid sequences to the sequence of an uncharacterized gene or, alternatively, for gene products (i.e., proteins) of known molecular or cellular function that have similar amino acid sequences to the gene product of an uncharacterized gene. For a general review of such techniques, see, e.g., Tatusov et al., 1997, Science 278:631-637; Koonin et al., 1998, Curr. Opin. Struct. Biol. 8:355-363. For example, computer algorithms and programs, such as the Basic Local Alignment Search Tool (BLAST) are well known in the art and are routinely used to compare different nucleic acid and amino acid sequences (see, in particular, Altschul et al., 1990, J. Mol. Biol. 215:403-410; Altschul et al., 1997, Nucleic Acids Res. 25:3389-3402; Tatusova and Madden, 1999, FEMS Microbiol. Lett. 174:247-250). Generally, such programs output results that specify a “percent identity” or “percent homology” to indicate the extent to which the two nucleotide or amino acid sequences are the same or similar. The fact that two nucleic acid or amino acid sequences are similar or “homologous” is then considered an indication that their corresponding genes or gene products have similar or equivalent molecular functions. However, identification of the cellular function does not necessarily follow, since a molecule identified as a “kinase” by sequence homology may have completely different roles in different cell types. Therefore, sequence homology is an imperfect indication of functional equivalence (see, Tatusov et al., 1997, Science 278:631-637; Koonin et al, 1998, Curr. Opin. Struct. Biol. 8:355-363).

While querying databases such as the GenBank database can provide useful information, often such information is inadequate because many novel genes do not have matches in such databases. It has recently been estimated that thirty percent of the proteins predicted to be in an organism bear no resemblance to any other sequence in the organism's own proteome or the proteome of any other organism (see, Ruben et et al., 2000, Science 287: 2204). Thus, based on such estimates, it is apparent that any effort to identify the function of a novel gene by sequence homology will necessarily fail on average at least thirty percent of the time due to the lack of any discernable sequence identity between the novel gene and any other gene in the database.

An example of an approach that has throughput limitations is a technique known as “reverse genetics.” In this technique, the phenotypes of known genetic mutations in an organism are observed (see, e.g., Sikorski and Boeke, 1991, Methods Enzymol. 194:302-318). Specifically, using in vitro mutagenesis and transformation techniques, mutant organisms and/or cell lines can be generated that contain a mutated version of a cloned gene of interest. Phenotypes of these mutants can then be examined to determine the cellular function of the gene in the cell line or organism.

An alternative approach, which is also known in the art, involves observing the physical association of gene products (e.g., proteins) with other proteins of known function, e.g., after purification over chromatographic columns or sedimentation velocity gradients, or using whole genome two-hybrid analysis. Proteins of unknown function are then presumed to be involved in the same cellular function as the protein or proteins with which they associate.

Other techniques are capable of providing insight on the molecular function, such as kinase or phosphatase activity, of a gene or gene product. Such techniques include, but are not limited to, the analysis and classification of structural properties (e.g., from x-ray crystallography), properties of spectral absorbance (such as absorption, fluorescence, circular dichroism, etc.) or cross-reactivity to monoclonal antibodies. For general discussions of such techniques see, e.g., Scopes and Smith, 1998, in Current Protocols in Molecular Biology, Vol. 2, Chapter 10: “Analysis of Proteins,” John Wiley & Sons, Inc. at pp. 10.0.1-10.0.20; Freifelder, 1982, Physical Biochemistry. Applications to Biochemistry and Molecular Biology, W. H. Freeman and Co. (San Francisco, Calif.); and Bartell et al, 1996, Nature Genetics 12:72-77. Although these techniques are invaluable for determining molecular function, additional techniques are required in order to elucidate the role of a particular gene or gene product in the cell.

Within the past decade, several technologies have made it possible to monitor the expression level of a large number of genetic transcripts within a cell at any one time. See, for example, Schena et al, 1995, Science 270:467-470; Lockhart et al., 1996, Nature Biotechnology 14:1675-1680; Blanchard and Hood, 1996, Nature Biotechnology 14:1649; Ashby et al., U.S. Pat. No. 5,569,588, issued Oct. 29, 1996; Velculescu, 1995, Science 270:484-487. In organisms for which the sequence of the entire genome is known, it is possible to analyze the transcripts of all genes within the cell. With other organisms, such as human, for which there is an increasing knowledge of the genome, it is possible to simultaneously monitor large numbers of the genes within the cell. Other technologies are known that permit high-throughput analysis of proteins, including two-dimensional gel electrophoresis (see, e.g., O'Farell, 1975, J. Biol. Chem. 250:4007-4021; Klose and Kobalz, 1995, Electrophoresis 16:1034-1059; Gygi and Aebersold, 1999, Methods Mol. Biol. 112:417-421; Gygi et al., 1999, Mol. Cell Biol. 19:1720-1730) and mass spectrometry (see, e.g., McCormack et al., Analytical Chemistry 69:767-776; Chait-B T, 1996, Nature Biotechnology 14:1544).

Previous applications of these technologies have included, for example, identification of genes that are up regulated or down regulated in various physiological states, particularly diseased states. Additional uses for transcript arrays have included the analyses of members of signaling pathways and the identification of targets for various drugs. See, e.g., International Patent Publication No. WO 98/38329 published on Sep. 3, 1998; Stoughton and Karp, U.S. Pat. No. 6,132,969; Stoughton and Friend, U.S. Pat. No. 5,965,352; Friend and Stoughton, U.S. patent application Ser. No. 09/303,082, filed Apr. 30, 1999; and U.S. patent application Ser. No. 09/334,328, filed Jun. 16, 1999. Transcript arrays have also been used to identify sets of cellular constituents, for example sets of genes or “gene sets,” in a single organism which co-vary in response to one or more different perturbations to the organism such as treatment with different drugs or modification in the activity of certain known proteins (see, for example, Stoughton et al., U.S. patent application Ser. Nos. 09/179,569, 09/220,142 and 09/220,275, filed on Oct. 27, 1998, Dec. 23, 1998 and Dec. 23, 1998, respectively). Individual members of a geneset are often associated with a common biological process or pathway. However, the determination that a gene is a member of a particular geneset does not, in itself, identify the particular function of that gene in any biological process or pathway associated with the particular geneset.

There continues to exist, therefore, a need for methods and compositions that can be used to rapidly characterize the function, particularly the cellular function, of large numbers of different genes and their gene products. In particular, there is a need for methods of rapidly comparing aspects of uncharacterized genes and gene products, such as their regulation, with those of genes and gene products having known cellular functions in order to identify functional homologs of the uncharacterized genes and gene products.

Discussion or citation of a reference herein shall not be construed as an admission that such reference is prior art to the present invention.

### 3. SUMMARY OF THE INVENTION

The present invention provides methods and compositions for characterizing the cellular function, including biological activities, of genes and their gene products. In particular, the methods and compositions of the present invention can be used to identify genes and gene products that have a common function in a cell or organism. For example, in particularly preferred embodiments, the methods and compositions of the invention are used to identify genes and gene products from different cells or organisms that are “functional homologs.” Such functional homologs, as the term is used herein, are understood to be genes and gene products that are functionally related and, in particular, carry out the same cellular function, e.g., in different organisms. Thus, the methods and compositions of the present invention provide information about the likely cellular role of an uncharacterized gene or gene product, such as a gene or gene product that has recently been isolated and sequenced, by identifying one or more candidate functional homologs of that gene or gene product having a known cellular function or activity. The cellular function or activity of the uncharacterized gene or gene product is likely to be the cellular function or activity of the one or more candidate functional homologs thus identified. Preferably, the methods and compositions of the present invention are used in conjunction with another technique, such as sequence alignment, gene replacement, or in vitro biochemical complementation, in order to identify the cellular function or activity of the uncharacterized gene or gene product.

An advantage of the present invention is that the techniques of the present invention are not dependent on the actual sequence homology between candidate genes. While sequence homology is useful in identifying functional homologs in some instances, sequence homology can actually hinder the identification of functional homologs in many instances. For example, consider a case where a particular phosphodiester (PDE) has been identified in a particular organism, perhaps because it has been shown to affect specific cellular activities in the organism. One may try to use sequence homology to determine the functional homolog of this specific PDE in a different organism. However, sequence homology in this instance will not be a reliable predictor of the functional homolog in the different organism because there exists a high degree of sequence homology throughout the PDE family. Thus, the presence of a degree of sequence homology between a PDE in a first organism and a PDE in a different organism does not necessarily prove that the two PDEs are functional homologs. Rather than relying on sequence homology, the methods of the present invention test for functional homologs by measuring the response of each of the PDEs in the different organism across a broad range of perturbations and by measuring the response of the known PDE in the first organism to the a similar or identical range of perturbations. Then, the functional homolog of the known PDE in the first organism is identified by finding the PDE in the different organism whose response to each of the broad range of perturbations is the most highly correlated to the corresponding response of the known PDE.

Another advantage of the present invention is that the cellular activity of a particular gene in one species can be determined using information on the same gene from another species in manner that is not dependent upon the sequence identity of the two genes. Yet another advantage of the present invention is that it can be sued to identify functional homologs across species in a high throughput manner to support industries such as the cross-specie gene annotation industry. Accordingly, the methods of the present invention can be used to rapidly populate, or check the accuracy of, important databases such as a commercial yeast-worm-fly database.

The methods of the invention involve comparing response profiles for different genes (or gene products) of interest and determining whether the two or more different genes (or gene products) are “co-regulated” over the responses. In particular, a first response profile is obtained or provided for a first gene (or gene product) of interest in a first cell or organism. The first response profile comprises measurements of the expression or abundance of the first gene or gene product in the first cell or organism in response to a plurality of different conditions or “perturbations,” such as graded exposure to one or more drugs. A second response profile is also obtained or provided for a second gene (or gene product) of interest in a second cell or organism. The second response profile likewise comprises measurements of the expression or abundance of the second gene or gene product in the second cell or organism in response to the same plurality of perturbations. The first and second response profiles are compared to determine whether the two or more different genes are co-regulated and, more specifically, whether the two or more response profiles are statistically correlated. Genes which are thus determined to be co-regulated are likely to be functionally related, i.e., are candidate functional homologs.

In various embodiments, the response profile may be obtained, e.g., by measuring gene expression, protein abundances, protein activities, amount of modification of a protein (e.g., modifications such as phosphorylation, cleavage, etc.) or protein activity, or a combination of such measurements. More generally, the response profile may be obtained by measuring expression levels of gene products, abundance of gene products, activity levels of gene products, or an amount of modification of gene products. Preferably, the first and second response profiles are obtained for genes from different cells or organisms and, most preferably from different species of organisms (or from cells of different species of organism). However, in other embodiments, the first and second response profiles may be obtained for different genes from the same organism. For example, the first response profile may be for a first gene in a first cell type or tissue type of an organism, and the second response profile can be for a second, different gene in a different cell type or tissue type of the same organism or, at least, of the same species of organism.

Applicants have discovered that genes and gene products that tend to respond together (i.e., are co-regulated) also tend to be functionally related in that they are members of a single coordinated response to certain perturbations to a cell or organism. Further, Applicants have also discovered that genes and gene products that are co-regulated, e.g., across different species of organisms and/or across different cell types, also tend to be functionally related. Thus, just as sequence homology between a first gene of unknown molecular function and a second gene of known molecular function can sometimes indicate the molecular function of the first gene, the co-regulation of genes and/or gene products can indicate their cellular functions. Unlike sequence homology, however, the co-regulation of different genes and gene products depends directly upon their cellular function and activity. Further, using the methods and compositions described herein, a skilled artisan can readily obtain and compare profiles for a large number of genes and gene products. Thus, the methods and compositions of the present invention provide high throughput methods of evaluating the function of genes and gene products that are well suited for the current demands.

In more detail therefore, the present invention provides methods for identifying a candidate functional homolog of a cellular constituent, said method comprising comparing a response profile for a cellular constituent of a first cell or organism to a response profile for a cellular constituent of a second cell or organism to determine whether said cellular constituents are co-regulated. The determination that said cellular constituents are co-regulated identifies said cellular constituent of said second cell or organism as a candidate functional homolog of said cellular constituent of said first cell or organism. In a preferred embodiment, said response profile for said cellular constituent of said first cellular constituent of said first cell or organism to said response profile for said cellular constituent of said second cell or organism is determined. In such embodiments, said cellular constituent of said second cell or organism is identified as a functional homolog of said cellular constituent of said first cell or organism if the correlation of said response profile for said cellular constituent of said first cell or organism to said response profile for said cellular constituent of said second cell or organism is, e.g., at least 50%, at least 75%, at least 80%, at least 85% or at least 90%. In preferred embodiments, said response profile for said cellular constituent of said first cell or organism comprises differential measurements of changes in said cellular constituents of said first cell or organism in response to a plurality of perturbations to said first cell or organism and/or said response profile for said cellular constituent of said second cell or organism comprises differential measurements of changes in said cellular constituent of said second cell or organism in response to a plurality of perturbations to said first cell or organism. Preferably said plurality of perturbations to said second cell or organism are the same as said plurality of perturbations to said first cell or organism.

In a particularly preferred embodiment of the invention, a perturbation subset is identified, said perturbation subset consisting of selected perturbations from said plurality of perturbations to said first cell or organism and wherein changes in cellular constituents of said first cell or organism in response to said selected perturbations are maximally informative. For example, in one embodiment, the selected perturbations of the perturbation subset are selected from said plurality of perturbations to said first cell or organism according to a method comprising: (a) clustering the perturbations of said plurality of perturbations to said first cell or organism into cluster groups according to similarities between responses of cellular constituents of said first cell or organism to the perturbations of said plurality of perturbations to said first cell or organism; and (b) selecting a representative perturbation from each of said cluster groups. In various embodiments the perturbations of said plurality of perturbations are clustered into at least 50, at least 100 (e.g., between 100-500) or at least 500 cluster groups. Thus, in various embodiments, the perturbation subset comprising at least 50, at least 100 (e.g., between 100-500) or at least 500 perturbations. In one embodiment, the representative perturbation selected from a particular cluster group is the perturbation of the particular cluster group which produces the most significant changes in said cellular constituents of said first cell or organism.

In various embodiments, the plurality of perturbations can comprise, e.g., exposure to one or more drugs, one or more mutations, one or more changes in protein activity or in protein abundances, changes in environmental conditions or exposure to one or more toxins. In various embodiments, the first cell or organism is different from the second cell or organism. For example, in certain embodiments the cellular constituents are preferably genes or gene products. In various embodiments the first cell or organism is a cell of a first species of organism and the second cell or organism is a cell of a second, different species of organism. In other embodiments, the first cell or organism is a first cell type of a first organism and the second cell or organism is a second, different cell type of a second organism (which can be the same organism or a different organism such as a different species of organism).

In other embodiments, the invention provides a computer system comprising a processor and a memory coupled to said process and encoding one or more programs. Specifically, the programs encoded by the memory of said computer system cause the computer system to execute the methods of the present invention; i.e., of (a) determining the correlation of a response profile for a cellular constituent of a first cell or organism to a response profile for a cellular constituent of a second cell or organism; and (b) determining whether said correlation is at least a threshold value (e.g., 50%, 75%, 80%, 85% or 90%), so that said cellular constituent of said second cell or organism is identified as a candidate functional homolog of said cellular constituent of said second cell or organism if said correlation is at least equal to said threshold value. In various embodiments, the programs encoded by the memory of a computer system of the invention can cause the processor to accept one or more of said response profiles entered into memory by a user or, alternatively, to read one or more of said response profiles into memory from a database. In certain embodiments, the programs further cause the processor to identify a perturbation subset consisting of a selected perturbation from a plurality of perturbations to said first cell or organism, wherein changes in cellular constituents of said first cell or organism in response to said selected perturbation are maximally informative. For example, in one embodiment, the programs cause the processor to select a perturbation of said perturbations subset by a method comprising: (a) clustering the perturbations of said plurality of perturbations to said first cell or organism into cluster groups according to similarities between responses of cellular constituents of said first cell or organism to the perturbations of said plurality of perturbations to said first cell or organism; and (b) selecting a representative perturbation from each of said cluster groups. In one aspect of this embodiment, the programs cause the processor to select said representative perturbations from each of said cluster groups by selecting, for each of said cluster groups a perturbation which produces the most significant changes in said cellular constituents of said first cell or organism.

The invention also provides, in other embodiments, a computer program product for use in conjunction with a computer having a processor and memory connected to the processor. The computer program product of the invention comprises a computer readable storage medium having a computer program mechanism encoded thereon, wherein the computer program mechanism can be loaded into the memory of the computer and cause the process to perform the methods of the present invention; i.e., the computer program mechanism can be loaded into the memory of the computer and cause the processor to execute the steps of: (a) determining the correlation of a response profile for a cellular constituent of a first cell or organism to a response profile for a cellular constituent of a second cell or organism; and (b) determining whether said correlation is at least a threshold value (e.g., 50%, 75%, 80%, 85% or 90%), so that said cellular constituent of said second cell or organism is identified as a candidate functional homolog of said cellular constituent of said second cell or organism if said correlation is at least equal to said threshold value. In various embodiments, the computer program mechanism can further cause the processor of the computer to accept one or more response profiles entered into memory by a user and/or read one or more response profiles from a database. In certain embodiments, the computer program mechanism can further cause the processor to identify a perturbation subset consisting of a selected perturbations from a plurality of perturbations to said first cell or organism, wherein changes in cellular constituents of said first cell or organism in response to said selected perturbations are maximally informative. For example, in one embodiment, the computer program mechanism can cause the processor to selected perturbations of said perturbations subset by a method comprising: (a) clustering the perturbations of said plurality of perturbations to said first cell or organism into cluster groups according to similarities between responses of cellular constituents of said first cell or organism to the perturbations of said plurality of perturbations to said first cell or organism; and (b) selecting a representative perturbation from each of said cluster groups. In one aspect of this embodiment, the computer program mechanism can cause the processor to select said representative perturbations from each of said cluster groups by selecting, for each of said cluster groups a perturbation which produces the most significant changes in said cellular constituents of said first cell or organism.

Each of these embodiments is described and enabled, in detail, in the sections hereinbelow, with reference to the following figures.

### 4. BRIEF DESCRIPTION OF THE FIGURES

FIG. 1 provides a flow chart illustrating an exemplary embodiment of the methods of the present invention.

FIG. 2 depicts an exemplary computer system that can be used to implement the methods of the present invention.

FIG. 3 depicts response profiles consisting of changes in expression levels of 1330 genes in the S. cerevisiae genome (horizontal axis) to 1490 different perturbation conditions (vertical axis) measured with a Genome Reporter Matrix (GRM). Both the genes and the perturbation conditions have clustered and reordered using the hierarchical clustering algorithm hclust, and the resulting cluster trees are shown on the left hand side (perturbation conditions) and top (genes) of the plot.

FIG. 4 shows the hierarchical cluster tree of the 1490 different perturbation conditions measured with the GRM in FIG. 3. The entire cluster tree structure for all 1490 different perturbations is shown on the left hand side of the figure with a dashed line indicating the user selected cutoff distance of 0.57. A region of this cluster tree is expanded on the right hand side of the figure illustrating nine exemplary cluster groups (indicated by solid dots) determined by the cutoff distance, and representative perturbation conditions (indicated by arrows) for each cluster group.

FIGS. 5A-5D compare gene-gene correlations among the 1330 genes measured in the GRM profiles depicted in FIG. 3. In particular, FIG. 5A plots the gene-gene correlations determined according to Equation 4 (Section 5.2.3, below) using the 1490 different perturbation conditions measured using the GRM assay, FIG. 5B shows the distribution of the gene-gene correlations depicted in FIG. 5A, FIG. 5C plots gene-gene correlations determined according to Equation 4 using only 106 perturbation conditions from perturbation subsets, and FIG. 5D shows the distribution of the gene-gene correlations depicted in FIG. 5C.

FIG. 6 is a gray-scale plot of the logarithmic level of gene expression ratios for 335 genes (horizontal axis) under 16 different perturbation conditions obtained with a GRM (indices 1-16 of the vertical axis) and using a transcript array (“GTM”; indices 17-32 of the vertical axis).

### 5. DETAILED DESCRIPTION

This section presents a detailed description of the present invention and its applications. In particular, Section 5.1 describes certain preliminary concepts useful in the further description of the invention, including the concepts of biological state and co-varying sets of cellular constituents. Section 5.2 provides a general description of the methods of the invention, while Section 5.3 describes certain, preferred analytical systems and methods for performing the methods described in Section 5.2. Sections 5.4 and 5.5 provide exemplary descriptions of particular embodiments of the data gathering steps that accompany the general methods of the invention described in Section 5.2. In particular, Section 5.4 describes methods of measuring cellular constituents and Section 5.5 describes various targeted methods of perturbing the biological state of a cell or organism that can be used, e.g., to obtain the response profiles evaluated in the methods of the present invention. Finally, certain exemplary applications of the methods and compositions of the invention are described in Section 5.6. The methods and compositions of the invention are also demonstrated by way of certain non-limiting examples which are presented in Section 6.

The description of the invention is by way of several exemplary illustrations, in increasing detail and specificity, of the general methods of the invention. The examples are non-limiting, and related variants that will be apparent to one skilled in the art are intended to be encompassed by the appended claims.

5.1. Introduction

The present invention relates to methods and compositions for determining (i.e., characterizing) the cellular function or activity of different cellular constituents. In particularly preferred embodiments, the methods and compositions of the invention are used to determine the cellular function or activity of different genes and/or their gene products (i.e., proteins). In more detail, the methods and compositions of the invention enable a user to compare response profiles of cellular constituents (e.g., genes or gene products) from different cells or organisms and determine the likelihood that the a cellular constituent in a first cell or organism is functionally related to, or a functional homolog of, a cellular constituent in a second cell or organism.

According to the present invention, the determination that a cellular constituent of a first cell or organism is a functional homolog of a particular cellular constituent of a second cell or organism is made by asking whether the cellular constituent is co-regulated in the first and second cell or organism.

To determine whether a cellular constituent is co-regulated in two different cells or organisms, a first response profile that includes a cellular constituent of interest in the first cell or organism and a second response profile that includes a cellular constituent of interest in the second cell or organism is measured after the respective cells or organisms have been subjected to a particular condition. In fact, several measurements are made for the first response profile. Each measurement represents the response of cellular constituents in the first cell or organism after the sample has been subjected to a different condition. Further, measurements for a second response profile are made. Each measurement for the second response profile represents the response of cellular constituents in the second cell or organism after the second cell or organism has been subjected to corresponding conditions used in the measurement made for the first response profile. Preferably, each of the measurements in the first and second response profile are differential measurements of the change in cellular constituent level that arise upon the introduction of the cell or organism to a particular condition. A cellular constituent is considered co-regulated if there is some form of statistical correlation in the measurement of the cellular constituent in the first and second response profiles.

To illustrate this technique, consider a cellular constituent x in X cells and a cellular constituent y in Y cells. Each measurement in the first response profile may be a measurement of the transcript level (or nucleic acid derived therefrom) of cellular constituent x after cell X has been subjected to a particular condition or perturbation. Thus, consider an instance where the set of perturbations {A} used includes three different perturbations, perturb_{—}1, perturb_{—}2, and perturb_{—}3. The first response profile will include three measurements, each made after a sample of X cells was subjected to a different perturbation in set {A}. The second response profile will include three corresponding measurements, each measuring the response of cellular constituents in a sample of Y cells after the cells have been subjected to a different perturbation in the set {A}. Generally speaking, in this example, cellular constituents x and y are considered co-regulated if the transcriptional level of cellular constituent x and y responded similarly to each of the perturbations in set {A}.

In some embodiments, a cellular constituent in the first response profile is considered coregulated with a cellular constituent in the second response profile when the response of the cellular constituent in the first and second response profiles is correlated across the set {A}. In one embodiment, a determination of whether cellular constituents are coregulated is made by calculating the correlation coefficient P_{xy} in accordance with Equation 4 in Section 5.2.3. Accordingly, as described in more detail in Section 5.2.3, cellular constituents x and y are considered coregulated when P_{xy} is at least 0.5.

Preferably, the methods of the present invention use large perturbations sets {A} as described in Section 5.2.2. Only one cellular constituent need be measured in the first cell or organism and second cell or organism. However, in typical applications of the present invention, several cellular constituents are measured in either the first cell or organism and quite possibly the second cell or organism because the identity of cellular constituents that may coregulate has not been determined. Thus, in some embodiments 5 or more cellular constituents are measured in the first cell or organism and/or the second cell or organism. In other embodiments, 20 or more cellular constituents are measured in the first cell or organism and/or the second cell or organism. In still other embodiments, 100 or more cellular constituents are measured in the first cell or organism and/or the second cell or organism. In yet other embodiments, 500 or more cellular constituents are measured in the first cell or organism and/or the second cell or organism.

A response profile comprises measurements or estimates of various aspects of the “biological state” of a cell or cells including, for example, the transcriptional state (e.g., mRNA abundances) the translational state (e.g., protein abundances) or the protein activity state. Such measurements are obtained under a plurality of different conditions, referred to herein as “perturbations” or “perturbation conditions,” such as exposure of the cell or cells to one or more drugs or to other compounds which are capable of having a biological effect on a cell or organism and which can therefore alter the biological state of the cell or organism. For example, the perturbations can include exposure to different toxins or exposure to different pesticides, including fungicides, herbicides and insecticides. Other exemplary perturbations can include mutations of one or more different genes (usually a gene or genes other than a gene whose expression or abundance is being measured) or changes in the expression or activity level of one or more proteins (again, usually proteins different from proteins whose abundances or activities are being measured). The different perturbations can also include different environmental conditions, including, but not limited to, growth or exposure to certain conditions of temperature, radiation, aeration or sunlight, or changes in the nutritional environment such as the presence or absence of certain amino acids, sugars or vitamins.

A “response profile,” as used herein, may therefore refer to the response of a particular cellular constituent in a cell type, cell culture or organism (“sample”) to a plurality of perturbations. Such perturbations include, for example, exposure of the sample to varying doses, concentrations or amounts of a particular drug or compound, exposure of the sample to varying doses concentrations or amounts of different drugs or compounds, and/or exposure of the sample to varying doses, concentrations or amounts of drug mixtures or compound mixtures. The exposure of a sample to several different types of perturbations may be referred to as a “gene plot.” Rather than being a “gene plot,” a response profile may be a “signature plot.” A signature plot refers to the response of a plurality of cellular constituents, such as mRNA levels or protein expression levels, in a sample to a particular perturbation. One of skill in the art will readily appreciate that response profiles of the first type, i.e., gene plots, are particularly useful in the methods of the present invention.

This section therefore provides definitions of concepts used to explain the present invention, including the concepts of biological function and activity and the concept of co-varying sets (including co-varying “genesets”). Next, a schematic and non-limiting overview of the methods of the invention is presented, in greater detail, in the following sections.

Although for simplicity, the description of the invention often makes reference to a single cell (e.g., “RNA is isolated from a cell exposed to a particular concentration of a drug”), it will be understood by those of skill in the art that, more often, any particular step of the invention will be carried out using a plurality of cells. Typically, these cells will be genetically identical cell derived, e.g., from a cultured cell line. Such similar cells are referred to herein as a “cell type.” Such cells are either from a naturally single celled organism such as yeast (e.g., S. cerevisiae) or bacteria (e.g., E. coli) or are derived from multi-cellular higher organisms including, for example, plant cells or animal cells, including cells of mammalian animals such as mice or rats, or from primates (e.g., monkeys and chimpanzees) including human cells. In fact, the cells used in the methods and compositions of the present invention may be cells derived from any organism.

5.1.1. Biological Function and Activity

The methods of the present invention involve comparing the effects of a plurality of different perturbations on a first cellular constituent (e.g., a gene or gene product) to the effect of said plurality of perturbations on a second cellular constituent. Cellular constituents, as the term is used herein, refer to components of the cell which can be used, either alone or, more typically, in combination with other cellular constituents, to characterize a cell's “biological state,” for example to characterize a cell's response to a particular drug, to a particular environmental change or condition, or to a particular mutation. In particularly preferred embodiments, the cellular constituents comprise genes and/or gene product (i.e., proteins) of a cell or organism.

In various embodiments therefore, the methods of the present invention can involve comparing measurements or estimates of the expression of one or more genes (such as measurements of certain mRNA abundances), comparing measurements or estimates of protein expression (such as measurements of certain protein abundances) or comparing measurements or estimates of certain protein activities.

As used herein, the term “cellular constituent” is not intended to refer to known subcellular organelles such as mitochondria, lysozomes, etc.

Typically, cellular constituents such as genes and their gene products will be associated with a particular activity or function (e.g., a particular “biological function” or “biological activity”) within a cell or organism. In particular, the biological function or biological activity of a cellular constituent, as the terms are used in the context of the present invention, are characterized by particular changes in the cellular constituent (e.g., changes in expression, abundance or activity) in response to particular perturbations to the cell or organism. As those skilled in the art will readily appreciate, cellular functions of cellular constituents characterized by changes in response to certain perturbations will generally be related to cellular functions of other cellular constituents characterized by similar changes in response to the perturbations. For example, and not by way of limitation, certain changes may be related, e.g., to particular biochemical activities (e.g., a reductase activity, a dehydrogenase activity or a kinase activity to a name a few). Thus, cellular constituents which have a similar or even an identical perturbation responses (i.e., which “co-vary” or which have “correlated” perturbation responses) are typically involved in a common biological function or activity and are likely to be “functionally related.” Further, cellular constituents such as genes and gene products from different cells or organisms, including cellular constituents from different species of organisms, that have similar or even identical perturbation responses (i.e., whose responses are “cross-correlated”) are also likely to be functionally related. Indeed, in some embodiments of the invention such cellular constituents can even have the same biological function or activity in their respective species of organism. Such cellular constituents are referred to herein as “functional homologs.”

5.1.2. Co-Varying Sets

In general, for any finite set of conditions, such as treatments with different concentrations of related compounds, cellular constituents will not all vary independently. Rather, there will be simplifying subsets of cellular constituents which typically change together, e.g., by increasing or decreasing their abundances and/or activities under some set of conditions or perturbations. Such cellular constituents are said to “co-vary” and are therefore referred to herein as co-varying cellular constituent sets or “co-varying sets.”

Further, the abundances and/or activities of individual cellular constituents are not all regulated independently. Rather, individual cellular constituents from a cell will typically share one or more regulatory elements with other cellular constituents from the same cell. For example, and not by way of limitation, in embodiments where the cellular constituents comprise genetic transcripts, the rates of transcription are generally regulated by regulator sequence patterns, i.e., transcription factor binding sites. Such cellular constituents are therefore said to be “co-regulated,” and comprise co-regulated cellular constituent sets or “co-regulated sets.”

As is apparent to one of skill in the art, those sets of cellular constituents which are co-regulated will, at least under certain conditions, co-vary. For example, and not by way of limitation, genes tend to increase or decrease their rates of transcription together when they possess similar transcription factor binding sites. Such a mechanism accounts for the coordinated responses of genes to particular signaling inputs. For example, see Madhani and Fink, 1998, Trends in Genetics 14:151-155; and Arnone and Davidson, 1997, Development 124:1851-1864. For instance, individual genes which synthesize different components of a necessary protein or cellular structure are frequently co-regulated. Also duplicated genes (see, e.g., Wagner, 1996, Biol. Cybern. 74:557-567) are frequently co-regulated and tend to co-vary to the extent that genetic mutations have not led to functional divergence in their regulatory regions. Further, because genetic regulatory sequences are modular (see, e.g., Yuh et al., 1998, Science 279:1896-1902), the more regulatory “modules” two genes have in common, the greater the variety of conditions under which they will co-vary in their expression levels. Physical separation between modules along the chromosome is also an important determinant since co-activators are often involved. Accordingly, and as is also apparent to one of skill in the art, the terms co-regulated set and co-varying set can be used interchangeably in the description of this invention.

5.2. Overview of The Methods of The Invention

The methods and compositions of the present invention enable a user to identify genes and gene products that are likely to be functionally related, including genes and gene products that are functional homologs such as orthologous genes and gene products that perform the same function in different species of organism. The methods involve analysis of biological responses (i.e., response profiles) which are obtained or provided from measurements of one or more aspects of the biological state of a cell or organism in response to a particular set or sets of perturbations. The perturbations may include, for example, drug exposure, targeted mutations or targeted changes in levels of protein activity or expression (see, for example, the specific exemplary perturbations that are described and enabled in Section 5.5, below). Other exemplary conditions or perturbations include changes in environmental conditions such as exposure to different conditions of temperature, radiation, sunlight, oxygen or aeration to name a few, as well as different nutritional conditions such as growth or incubation of the cell or organism in the presence or absence of particular nutrients (e.g., one or more particular amino acids and/or sugars). Still further, exemplary perturbations also include exposure of the cell or organism to one or more toxins including, but not limited to, exposure to pesticides (including, e.g., fungicides or insecticides) or herbicides.

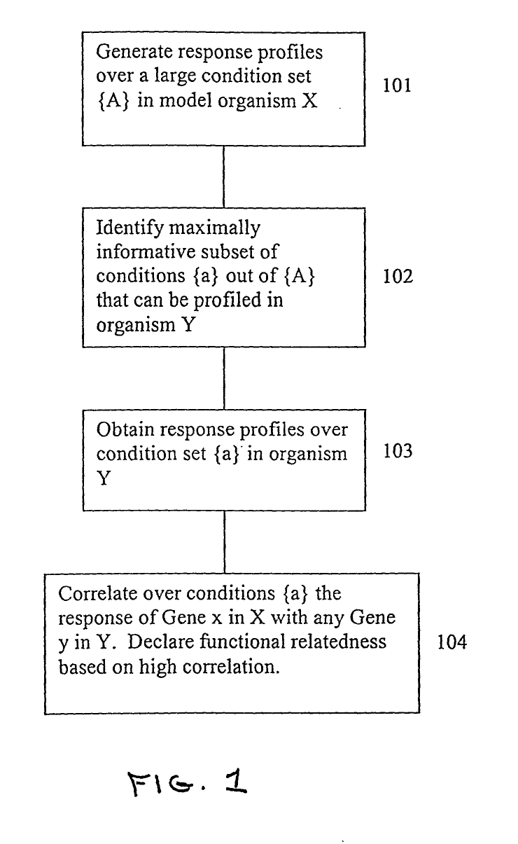

Particular aspects of the biological state of a cell, such as the transcriptional state, the translational state or the activity state are obtained or measured (e.g., according to the exemplary methods described in Section 5.4, below) in response to the plurality of perturbations. Preferably, the measurements are differential measurements of the change in cellular constituents in response, e.g., to a drug at certain concentrations and times of treatment. The collection of these measurements, which are optionally graphically represented, are called herein the “pertubation response” or “drug response” or, alternatively, the “response profile.” In preferred embodiments of the invention, a plurality of different response profiles are obtained or provided for a plurality of different perturbations or for a plurality of cellular constituents. Specifically, perturbation responses are preferably obtained or provided for cellular constituents (e.g., gene transcripts and/or gene products) having an unknown function as well as for a one or more cellular constituents (e.g., gene transcripts and/or gene products) that have a known function and are suspected of being functionally related to one or more of the cellular constituents having an unknown function. An overview of an exemplary embodiment of the methods of the invention is shown in FIG. 1. These methods are described, in detail, hereinbelow.

5.2.1. Generating Response Profiles

In more detail, a first response profile is first obtained or provided (FIG. 1, step 101) for a particular cellular constituent (e.g., a particular gene or gene product) of interest (referred to herein as cellular constituent x) in a first cell or organism (referred to herein as X) under some particular set of perturbations. In particular, the set of perturbations for which a response profile is obtained is referred to herein as the “perturbation set,” and denoted {A}. Because the methods and compositions of the invention are preferably used in the high throughput analysis of genes and gene products, response profiles are, in fact, most preferably obtained or provided simultaneously for a plurality of different cellular constituents under the perturbation set {A}, e.g., using a microarray as described in Section 5.4.2. In such embodiments, the response profiles are preferably obtained or provided for different cellular constituents, particularly for different genes or gene products of the same cell or organism. In one embodiment, the value of the expression or abundance of the cellular constituent x used in the analytical methods of the invention is expressed relative to some baseline value of the expression or abundance of x. For example, in some embodiments, the expression or abundance of x under a particular condition or perturbation i is expressed as the ratio of the absolute expression or abundance of x under the particular condition or perturbation i to the absolute expression or abundance of x under a “baseline” or “neutral” condition (e.g., a condition in which the cell or organism is not perturbed). Exemplary neutral or baseline conditions include, but are not limited to, conditions of optimal growth for the cell or organism or conditions that are typical of the natural environment of the cell or organism. In another embodiment, the value of the expression or abundance of the cellular constituent x used in the analytical methods of the invention is the absolute measured amount of the expression or abundance of the cellular constituent.

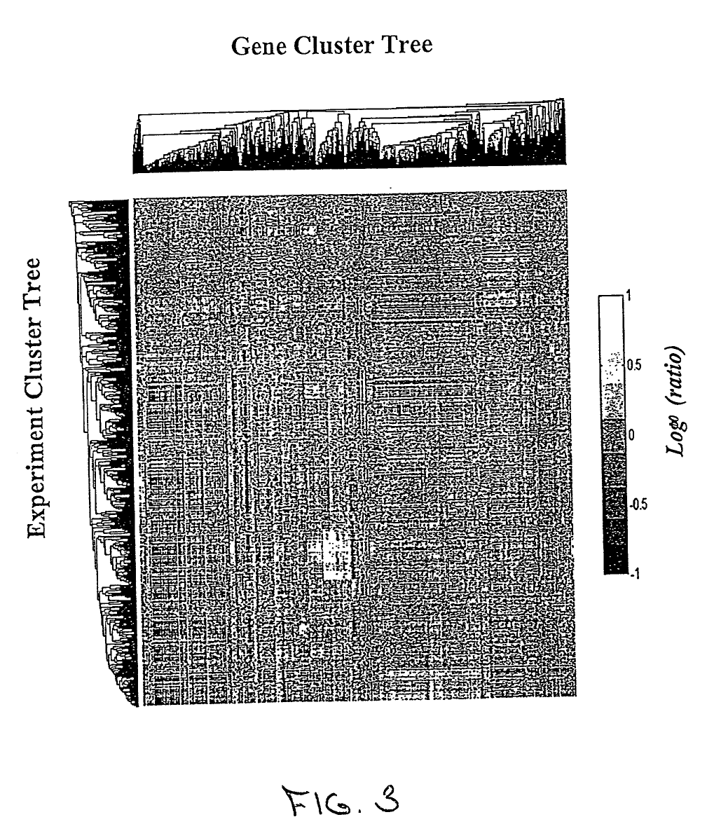

For example, and not by way of limitation, FIG. 3 illustrates response profiles of particular genes of the yeast S. cerevisiae under 1490 different perturbation conditions measured using the Genome-Reporter Matrix (“GRM”) of Dimster-Denk et al. (1999, J. Lipid Res. 40:850-869). In more detail, each row of the plot shown in FIG. 3 represents the response of a set of yeast genes to one of 1490 different perturbations to yeast cells, i.e., the signature plot. The exemplary perturbations include, but are not limited to, treatment of the cells with different chemical compounds (including vanillin, ethidium bromide, fluorouracil, tetracycline, methotrexate, pentenoic acid, azoxystrobin, prochloraz, sulfacetimide, sulfamethoxazole, sulfisoxazole, sulfanilamide and asulam to name a few) at various concentrations and targeted mutations to a number of different genes (including pet117, qcr2, fks1, phd1 and sod1, to name a few). Each column of the plot therefore represents the response profile for a particular gene of the S. cerevisiae genome, i.e., the gene plot.

Optionally, both the cellular constituents and the perturbations can be ordered and displayed according to similarity clustering as described, e.g., in U.S. patent application Ser. Nos. 09/179,569; 09/220,142 and 09/220,275 filed on Oct. 27, 1998, Dec. 23, 1998 and Dec. 23, 1998, respectively. Methods of cluster analysis that can be used to reorder cellular constituents and/or response profiles are also described in U.S. patent application Ser. No. 09/428,427 entitled “METHODS OF USING CO-REGULATED GENESETS TO ENHANCE DETECTION AND CLASSIFICATION OF GENE EXPRESSION PATTERNS” by Stephen H. Friend, Roland Stoughton and Yudong He and filed on Oct. 27, 1999. For example, in FIG. 3 both the columns (i.e., the genes) and the rows (i.e., the perturbations) have been clustered by a hierarchical agglomerative clustering technique using the hclust clustering algorithm (MathSoft, Seattle, Wash.) and as explained below. While not necessary to practice the methods of the invention, such “two-dimensional clustering” is often preferable since it provides a convenient and useful visualization means for identifying correlated genes and/or perturbations in subsequent analytical steps of the invention.

5.2.2. Identification of a Perturbation Subset

Preferably, the number of different conditions or perturbations contained in the perturbation set {A} is very large. In preferred embodiments, {A} includes at least 10 different conditions or perturbations, in more preferred embodiments, {A} includes at least 50 different conditions or perturbations, in even more preferred embodiments, {A} includes at least 100 different conditions or perturbations, in still more preferred embodiments, {A} includes at least 500 different conditions or perturbations, and in the most preferred embodiment, {A} includes at least 1000 different conditions or perturbations. However, in order to practice the methods of the invention most efficiently, the response profiles obtained for perturbation set {A} are preferably evaluated (as depicted in optional step 102 of FIG. 1) and a “perturbation subset,” denoted herein as {a}, is selected. Specifically, the perturbation subset {a} consists of those perturbations or conditions in the perturbation set {A} for which the profiles of gene x, or in more preferred embodiments of a plurality of genes, in the cell or organism X are maximally informative (e.g., strongest and, preferably, most diverse).

For example, if several of the profiles obtained for the cell or organism X are closely correlated with each other, then typically only one of the conditions or perturbations from this group is selected for further analysis according to the methods of the present invention. Many techniques of analysis are known in the art that can be used to assess the similarity and/or correlation between two or more different profiles. For example, in those embodiments in which levels of expression or abundance are obtained for only a single cellular constituent (i.e., for a single gene or gene product, x), the similarity of the expression or abundance of x under two or more different conditions (e.g., the conditions i and j) can be evaluated simply by comparing the relative values of xᵢ and xⱼ wherein xᵢ and Xⱼ denote the measured or estimated levels of expression or abundance of x under the conditions i and j, respectfully. As a particular example, and not by way of limitation, by comparing the values of xᵢ and xⱼ using the equation Dᵢⱼ=(Xᵢ²−xⱼ²)^{½}, one skilled in the art will readily appreciate that responses of x that are similar under the conditions i and j will have values of Dᵢⱼ that are equal to or near zero, whereas responses of x that are dissimilar under the conditions i and j will cause Dᵢⱼ to be large. A more preferable equation for comparing values xᵢ and xⱼ is the equation Dᵢⱼ=|xᵢ²−xⱼ²|². Here, responses of x that are similar under the conditions i and j will have values of Dᵢⱼ that are equal to or near zero. Furthermore, because discrepancies between the square of xᵢ and the square of Xⱼ are themselves squared in this equation, responses of x that are dissimilar under the conditions i and j will cause Dᵢⱼ to become very large.

As noted above, however, response profiles are preferably obtained and compared simultaneously for a plurality of genes. In such embodiments, the correlation of different profiles is evaluated by using cluster analysis methods, e.g., as described in U.S. patent application Ser. Nos. 09/179,569; 09/220,142 and 09/220,275 filed on Oct. 27, 1998, Dec. 23, 1998 and Dec. 23, 1998, respectively. Methods of cluster analysis that can be used to evaluate profiles in the perturbation set {A} are also described in U.S. patent application Ser. No. 09/428,427 entitled “METHODS OF USING CO-REGULATED GENESETS TO ENHANCE DETECTION AND CLASSIFICATION OF GENE EXPRESSION PATTERNS” by Stephen H. Friend, Roland Stoughton and Yudong He and filed on Oct. 27, 1999.

Briefly, and in a preferred but non-limiting embodiment in which response profiles are compared simultaneously for a plurality of cellular constituents (e.g., for K different cellular constituents in which K is a positive integer with a value greater than one), the similarity between the responses of a cellular constituent to two perturbations i and j can be evaluated by means of a distance metric such as:

Dᵢⱼ=1−|ρᵢⱼ| (Equation 1)

where the correlation coefficient ρᵢⱼ is provided by the equation:

$${ρ}_{ij} = \frac{{\sum }_{k} {x}_{ik} {x}_{jk}}{{( {\sum }_{k} {x}_{ik}^{2} {\sum }_{k} {x}_{jk}^{2} )}^{1 / 2}} \qquad ( Equation 2 )$$

In Equation 2, xᵢₖ refers to the expression level (absolute or normalized) of the cellular constituent xₖ in response to the perturbation i. The expression levels are summed over the cellular constituent index; i.e., k=1 to K. In certain aspects of such embodiments, the summation over the cellular constituent index can be restricted. For example, the summation can be restricted to those cellular constituents for which xᵢₖ or xⱼₖ is different from zero. In another example, the summation is restricted to those cellular constituents that have a statistically significant response to the perturbation(s) i and/or j or, alternatively, to those cellular constituents having a response to the perturbation(s) i and/or j that is above some minimum or threshold value selected by a user.

In still other embodiments, the similarity between two or more different response profiles is evaluated according to other mathematical techniques well known to those skilled in the art. For example, in one preferred alternative embodiment the similarity between two or more different response profiles is determined using Shannon mutual information theory as described, e.g., by Shannon and Weaver, 1998, Neural Computation 10:1731-1757).

Once values for a distance metric Dᵢⱼ are obtained, clustering of the different conditions or perturbations is done, for example, according to hierarchical agglomerative clustering methods that are well known to those skilled in the art. In one embodiment, clustering of the different conditions or perturbations is done using the S-Plus (MathSoft, Seattle, Wash.) hclust algorithm. In alternative embodiments, clustering is done, e.g., by K-Means (see, in particular, Hartigan, 1975, Clustering Algorithms, Wiley & Sons, New York) or using Self-Organizing Maps as described, e.g., by Kohonen (1995, Self Organizing Maps, Springer, Berlin). In such embodiments, the number of clusters must be chosen by a user. In particular, the number of cluster groups is pre-specified by a user in embodiments wherein methods such as K-Means clustering or Self-Organizing Maps are utilized. Alternatively, in embodiments, such as the hclust algorithm, that generate a “clustering tree,” the number of cluster groups can be set by selecting a similarity threshold in the clustering tree (e.g., by selecting a “threshold” value for Dᵢⱼ in Equation 1, above). Preferably, the number of cluster groups is selected to be equal to the number of conditions or perturbations that will be profiled in the comparison organism.

The exact number of cluster groups selected in particular embodiments of the invention will depend both on the need for accuracy in the gene-gene correlations determined and on the need to economize the number of experiments performed in the methods of the invention. In particular, the number of cluster groups is preferably large enough that gene-gene correlations determined for a representative perturbation from each cluster group are identical to, or at least substantially identical to, gene-gene correlations determined for all of the perturbations of the original perturbation set {A}. In this regard, one embodiment of the present invention provide a correlation coefficient cut-off of 0.5 or greater. In a more stringent embodiment, a correlation cut-off of 0.7 or greater is applied.

The number of clusters is preferably sufficiently small so that the methods of the invention can be readily practiced using a relatively small number of perturbation experiments since such experiments may be expensive and time consuming. Thus, for example, the number of cluster groups is preferably at least 50 and, more preferably, between 100 and 500. One skilled in the art will be able to select appropriate numbers of cluster groups for particular embodiments in view of the teaching provided herein, including the teaching of the Example presented in Section 6, below.

Once perturbations have been clustered and/or individual cluster groups are identified, a single, representative perturbation is preferably selected from each cluster group (e.g., by a user) for inclusion in the perturbation subset {a}. Preferably, the single perturbation selected from a cluster group is the perturbation producing the most significant changes in the cellular constituents xₖ. For example, the individual perturbations i in each cluster group can be ranked according to the metric Sᵢ, wherein

$${S}_{t} = {\sum }_{k} {( \frac{{x}_{ik}}{{σ}_{k}} )}^{2} \qquad ( Equation 3 )$$

and σₖ is the actual or expected root mean squared (“RMS”) measurement error in the cellular constituent xₖ in response to the perturbation i. Thus, for example, the perturbation in a particular cluster group for which Sᵢ has the largest value in that group can be selected as the single representative perturbation for inclusion in the perturbation subset. In still other embodiments, the representative perturbation can be selected from each cluster set, e.g., having the most changes xᵢₖ that are above a certain threshold (e.g., the most changes that are at least two-fold or, alternatively, the most changes by at least an order of magnitude).

In some embodiments, the perturbation subset {a} will comprise at least some perturbations to the organism X that cannot be realized with a second cell or organism of interest (i.e., with a second, different cell or organism Y). For example, in some embodiments the perturbations to the cell or organism X may include mutations to a particular gene or genes of the cell or organism X for which an analogous gene or genes have not yet been identified in the second cell or organism Y. However, because the methods of the invention involve comparing response profiles from different cells or organisms, the perturbation subset {a} most preferably consists of perturbations to the cell or organism X that can also be accomplished or realized for a second cell or organism of interest (i.e., for y). For example, the perturbations of the perturbation subset {A} can be selected so that the perturbation set consists only of perturbations that can be accomplished or realized in each cell or organism of interest (i.e., in each cell or organism whose response profiles are to be compared according to the methods of the invention). Alternatively, the perturbations of the perturbation set {A} can include both perturbations that can be realized in each cell or organism of interest and perturbations that cannot be realized in each cell or organism of interest. Preferably in such an embodiment, only those perturbations in the perturbation set {A} that can be realized in each organism of interest is then analyzed in the selection of the perturbation subset {a}.

5.2.3. Cross-Correlation of Cellular Constituents

The methods of the present invention involve comparing a response profile from a first cell or organism to a response profile from a second cell or organism. Accordingly, a response profile is also obtained or provided (FIG. 1, step 103) for a particular cellular constituent (e.g., a particular gene or gene product) of interest (referred to herein as y) in a second cell or organism (referred to herein as Y) under a particular set of perturbations. As noted above, the methods and compositions of the present invention are preferably used in the high throughput analysis of genes and gene products. Accordingly, most preferably response profiles are obtained or provided for a plurality of cellular constituents (e.g., for a plurality of different genes or gene products) in the second cell or organism under the particular set of perturbations.

Preferably, the two cells or organisms X and Y are different cells or organisms. For example, in one particularly preferred embodiment the first cell or organism X is a cell or cell sample from a first species of organism and the second cell or organism Y is a cell or cell sample from a second, different species of organism. In certain other preferred embodiments, the first and second cell or organism are different cells or cell samples from the same species of organism. For example, in one embodiment, the first cell or organism X is a cell or cell sample from a first strain of a particular species of organism and the second cell or organism Y is a cell or cell sample from a second, different strain of the same particular species of organism. In another exemplary embodiment, the first cell or organism X is a particular cell-type of a particular species of organism and the second cell or cell sample Y is a different cell-type of the same particular species of organism. In yet another exemplary embodiment, the first cell or organism X is a cell or tissue sample from a particular type of tissue of a particular species of organism and the second cell or organism Y is a cell or tissue sample from a different type of tissue of the same particular species of organism.

The set of perturbations for which responses are obtained or provided for cellular constituents , of the second cell or organism Y preferably consist of the same perturbations for which responses are obtained or provided for cellular constituents of the first cell or organism X. That is, the set of perturbations for which responses are obtained or provided for cellular constituents of the second cell or organism Y are preferably members of the perturbation set {A}. More preferably the set of perturbations for which responses are obtained or provided for cellular constituents y of the second cell or organism Y are preferably members of the perturbation subset {a}. In fact, most preferably the set of perturbations for which a response profile is obtained or provided for cellular constituents y of the second cell or organism Y include all of the perturbations that are members of the perturbation subset {a}.

A response profile having been obtained or provided for cellular constituents from cells or organisms X and Y. the methods of the invention can then be used to determine whether particular cellular constituents x and y from the cells or organisms X and Y, respectively, are candidate functional homologs. Specifically, the methods of the invention can be used to evaluate the co-regulation of x and y across a common set of conditions or perturbations, most preferably across the perturbation subset {a}. For example, the similarity (i.e., correlation) of the response profile of the genes or gene products x and y can be evaluated by means of the equation:

$${ρ}_{xy} = \frac{{\sum }_{i} {x}_{i} {\sum }_{i} {y}_{i}}{{( {\sum }_{i} {x}_{i}^{2} {\sum }_{i} {y}_{i}^{2} )}^{1 / 2}} \qquad ( Equation 4 )$$

in which xᵢ and yᵢ denote respective changes in expression, abundance, activity levels or amount of modification of the gene products corresponding to the cellular constituents x and y, respectively, under the condition or perturbation i. Those cellular constituents, x and y, for which the correlation p_{xy} is particularly high are then identified as being functionally related and are thus determined to be candidate functional homologs. Preferably, the candidate functional homologs identified according to the methods of the invention have a correlation P_{xy} that is at least 0.5 (i.e. at least 50%). More preferably, the candidate functional homologs identified according to the methods of the invention have a correlation that is at least 0.75 (i.e., at least 75%), 0.8 (i.e., at least 80%) or at least 0.85 (i.e., at least 85%). In fact, the candidate functional homologs identified according to the methods of the invention most preferably have a correlation that is at least 0.9 (i.e., at least 90%).

Other forms of determining correlation between two datasets, besides the correlation coefficient of Equation 4 are well known in the art. Indeed, any statistical method for determining the probability that two datasets are related may be used in accordance with the methods of the present invention in order to identify functional homologs. Correlation based on ranks is also possible, where xᵢ and yᵢ are the ranks of the measurement in ascending or descending numerical order. See e.g., Conover, Practical Nonparametric Statistics, 2^{nd} ed., Wiley, (1971). Shannon mutual information also can be used as a measure of similarity. See e.g., Pierce, An Introduction To Information Theory: Symbols, Signals, and Noise, Dover, (1980).

From Equation 4, it will be appreciated that the same conditions i are preferably applied to samples X and Y. However, there is no requirement that each condition i applied to X and Y be identical. For instance, p_{xy} could be computed using the equation:

$${ρ}_{xy} = \frac{{\sum }_{iX} {x}_{iX} {\sum }_{iY} {y}_{iY}}{{( {\sum }_{iX} {x}_{iX}^{2} {\sum }_{iY} {y}_{iY}^{2} )}^{1 / 2}} \qquad ( Equation 5 )$$

where iX is a perturbation applied to X and iY is the corresponding perturbation applied to Y Equation 5 allows for instances where, for example, iX is the exposure of X to 50 mM of a compound N for 30 minutes whereas iY is the exposure of Y to 73 mM of compound N for 33 minutes. In such instances, although perturbation iX and iY are somewhat different, useful information can be derived from the computation of Equation 5.

Furthermore, it will be appreciated that calculated response values can be estimated based on measured response values xᵢ and yᵢ. For example, if xᵢ and yᵢ were measured using the perturbations 25 mM exposure to compound N, 75 mM exposure to compound N, and 100 mM exposure to compound N, a response to exposure to 50 mM compound N can be estimated from the observed data using a data reduction technique such as least squares analysis. See, e.g., Data Reduction and Error Analysis for the Physical Sciences, Bevington & Robinson, 2^{nd} Ed., McGraw-Hill, Boston, Mass., 1969. This estimated response value can then be used in either Equation 4 or 5.

In many embodiments of the invention, measurement errors and/or other artifacts (e.g., signal noise) may distort correlation values obtained according to Equation 4 (see section 5.2.3). For example, genes or gene products that have very weak or low levels of expression or abundance can have large correlation values even though the genes or gene products may not, in fact, be functional homologs. Alternatively, if the levels of expression or abundance have large measurement errors associated with them, the correlation calculated according to Equation 4 (section 5.2.3) may be small even though the genes or gene products actually are functional homologs. Accordingly, in preferred embodiments of the invention, a ranking formula, similar to the ranking formula described in Equation 3, above, is used to distinguish cellular constituents that generally have weak responses from those cellular constituents having strong responses. An exemplary, preferred ranking formula is of the form

$${S}_{k} = \frac{1}{N} {\sum }_{i} {( \frac{{x}_{ki}}{{σ}_{k}} )}^{2} \qquad ( Equation 6 )$$

wherein xₖᵢ denotes the response (e.g., the level of expression or abundance) of the cellular constituent xₖ to perturbation i of the response profiles (i.e., of the perturbation set or, more preferably, of the perturbations subset). σₖ is the actual or expected RMS measurement error in the xₖᵢ. N denotes the total number of perturbations. In typical embodiments, where the error in the measured signal is due to random noise, the ranking function of Equation 6, above, is distributed as χ² with N degrees of freedom. Such a distribution can be readily analyzed, e.g., using the chi-square probability function (i.e. the P-value) which is well known to those skilled in the art (see, e.g., Meyer, Data Analysis for Scientist and Engineers, John Wiley, New York, 1975). Those cellular constituents that have large values of Sₖ that are unlikely to be generated by random noise (e.g., that are associated with small P-values such as P-values less than 0.01 or less than 0.001) will produce correlations that are most likely to reflect the actual function of the cellular constituents. Thus, in preferred embodiments of the invention, only those cellular constituents having unlikely values of Sₖ (i.e., values of Sₖ that are associated with small P-values such as the P-values recited supra) are evaluated in the methods of the invention (e.g., using Equation 4, section 5.2.3).

5.3. Implementation Systems and Methods

The analytical methods of the present invention are preferably implemented by means of an automated system such as a computer system. Accordingly, this section describes exemplary computer systems which may be used to perform the methods of the present invention, as well as methods and programs for operating such computer systems.

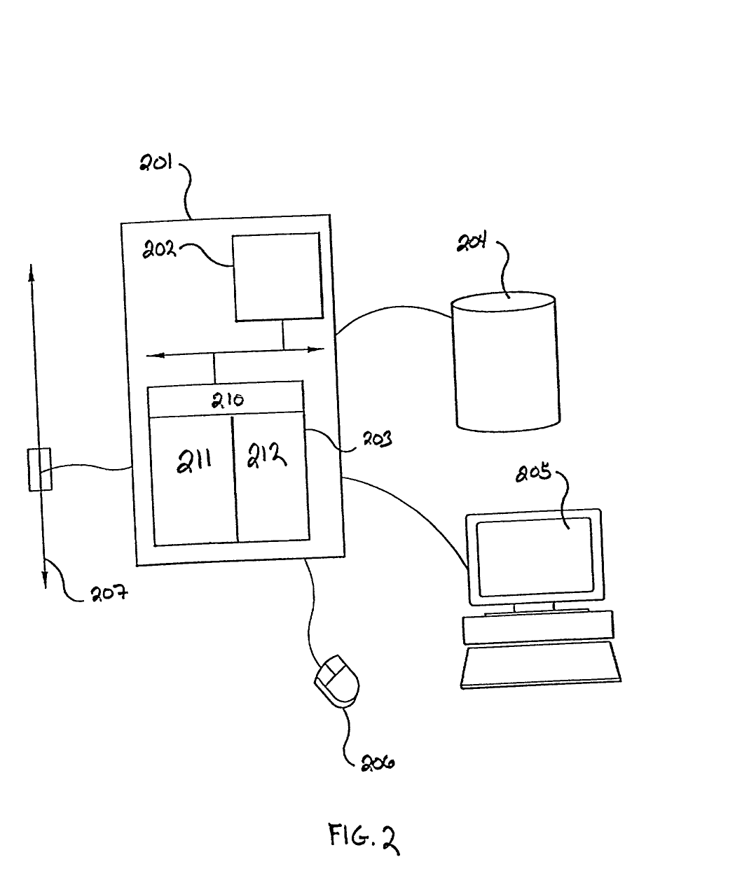

FIG. 2 illustrates an exemplary computer system suitable for implementing the analytical methods of the present invention. The computer system (201) comprising internal components linked to external components. The internal components of this exemplary computer system include a processor element (202) interconnected with a memory (203). For example, the computer system can comprise an Intel Pentium®-based processor of 200 MHz or greater clock rate and with 32 Mb or more of memory. The external components include one or data mass storage means (204). This data storage means can be, e.g., one or more hard disks (which are typically packaged together with the processor and the memory). Typical hard disks which can be used in such a computer system have a storage capacity of 1 Gb or more. Other means of data storage can also be used such as CD-ROM, floppy disk, or tape (e.g. DAT tape). Other exemplary external components can include a user interface device (205) such as a monitor, together with an inputting device (206) which can be, e.g., a keyboard and/or a “mouse.” A printing device (not illustrated) can also be attached to the computer system.

Typically, a computer system (201) of the invention is also linked to a network link (207), which can be, e.g., an Ethernet link to one or more local computer systems, to one or more remote computer systems or to one or more wide area communication networks such as the Internet. The network allows the computer system to share data and processing tasks with other computer systems. Thus, the methods of the invention can be implemented by means of a plurality (i.e., two or more) computer systems that are connected on a network as well as by a single computer system.

Loaded into the memory during operation of the computer system are several software components which are both standard in the art and special to the present invention. These software components collectively cause the computer system to function according to the methods of the present invention. Typically, the software components are stored on data storage means (204) and loaded into the memory during operation. For example, software component 210 represents an operating system which is responsible for managing the computer system. The operating system can be, for example, of the Microsoft Windows family, such as Window95, Windows98, WindowsNT or Windows2000. Alternatively, the operating system can be a Macintosh operating system or a UNIX operating system such as LINUX.

Software component 211 represents common language and functions that are preferably present on the computer system to assist programs implementing methods that are specific to the present invention. For example, many high or low level computer languages can be used to program the analytical methods of the invention. Instruction can be interpreted during run-time or they can be interpreted before run time (i.e., “compiled”) for later execution. Preferred languages include, but are not limited to, C, C++ and, less preferably, FORTRAN or JAVA. Most preferably, the methods of the present invention are programmed in mathematical software packages that allow symbolic entry of equations and high-level specification of processing, including algorithms to be used. Such software packages are preferable since they typically free a user of the need to procedurally program individual equations or algorithms. Mathematical software packages which may be used in the computer systems of the invention include, but are not limited to, Matlab from Mathworks (Natick, Mass.), Mathematica from Wolfram Research (Champaign, Ill.), S-Plus from MathSoft (Seattle, Wash.).

Finally, software component 212 represents the analytical methods of the invention as programmed, e.g., in a procedural language or symbolic package. In particular, the analytical software component preferably includes one or more programs that cause the processor to execute steps of accepting a response profile for a first cellular constituent x and for a second cellular constituent y, and comparing those profiles (e.g., according to the cross-correlation methods described in Section 5.2.3 above) and determining whether the two cellular constituents are candidate functional homologs. In one embodiment, the response profiles can be entered directly into the memory by a user, e.g., using the keyboard. However, in another embodiment the analytical software causes the processor to load response profiles into the memory from a database of response profiles.

In one particularly preferred embodiment, the analytical programs cause the processor to accept a response profile for a cellular constituent (e.g., a gene or gene product) of unknown biological function. The programs then cause the processor to load into memory response profiles for a plurality of cellular constituents from a database (e.g., a database of response profiles for cellular constituents of known biological function or activity). The programs cause the processor to compare, according to the methods of the invention, the response profile for a cellular constituent from the database to the response profile for the cellular constituent of unknown function and to determine whether any of the cellular constituents whose response profile is in the database are candidate orthologs of the cellular constituent of unknown function.

In preferred embodiments, the analytical software component also includes one or more programs, e.g., for clustering both perturbation conditions and/or cellular constituents (e.g., as discussed in Section 5.2.1 above) to facilitate data analysis according to the analytical methods of the present invention. The analytical software component can also include one or more programs that cause the processor to accept a response profile for one or more cellular constituents for a full perturbation set and identify a reduced perturbation set according to the methods of the invention (see, e.g., Section 5.2.2 above).

As mentioned supra, the computer systems of the present invention preferably receive one or more response profiles from a database. Such databases are also understood to be part of the present invention. In particular, such a database will preferably contain entries for one or more cellular constituents (e.g., for one or more genes or gene products). For example, in one preferred embodiment, the database includes an entry of all known genes of one or more organisms (e.g., for yeast such as S. cerevisiae or for human). The entry for each cellular constituent preferably includes a response profile for the cellular constituent to a plurality of different perturbations. However, the entry for each cellular constituent can further include other information about the cellular constituent that may be useful to a user when identifying candidate orthologs. For example, in embodiments wherein the cellular constituents are genes or gene products, the database entries can also contain the nucleic acid or amino acid sequence of each gene or gene product. In other preferred embodiments, the database entry for each cellular constituent can also include cross-correlation values, determined, e.g., according to Equation 6 above and indicating the correlation of a response profile for the cellular constituent to the response profile for one or more other cellular constituents (for which, preferably, there are also entries in the database). Finally, the entry for each cellular constituent in a database also preferably contains information that describes the cellular function and/or activity, if known, for the cellular constituent.

The analytical systems of the invention also include computer program products that contain one or more of the above-described software components such that the software components can be loaded into the memory of a computer system. Specifically, a computer program product of the invention includes a computer readable storage medium having one or more computer program mechanisms embedded or encoded thereon in a computer readable format. The computer program mechanisms encode, e.g., one or more of the analytical software components described above, which can be loaded into the memory of a computer system and cause the processor of the computer system to execute the analytical methods of the present invention.

Both the computer program mechanisms and the databases of the present invention are preferably stored or encoded on a computer readable storage medium. Exemplary computer readable storage media are discussed above and include, but are not limited to: a hard drive which can be, e.g., an external or internal hard drive of a computer system of the invention or a removable hard drive; a floppy disk; a CD-ROM; or a tape such as a DAT tape. Other computer readable storage media that can be used for the computer program mechanisms and databases of the present invention will also be apparent to those skilled in the art.

Alternative, equivalent systems and methods for implementing the analytic methods of this invention will also be apparent to those skilled in the art and are intended to be comprehended within the accompanying claims. In particular, alternative program structures for implementing the methods of this invention will be readily apparent to those of skill in the art and are also considered part of the present invention.

5.4. Measurement Methods

Responses such as drug responses are obtained or provided for use in the present invention by measuring the cellular constituents changed by a perturbation, such as exposure to one or more drugs or targeted mutations to one or more genes. These measurements can be of any aspect of the biological state of a cell or organism. For example, the measurements can be measurements of the transcription state (in which RNA abundances are measured), the translation state (in which protein abundances are measured) or the activity state (in which protein activities are measured) to name a few. The measurements can also be measurements of mixed aspects of the biological state, for example, in which the activities of one or more proteins are measured along with RNA abundances (i.e., levels of gene expression). This section describes certain exemplary methods for measuring the cellular constituents in perturbation responses. However, the methods and compositions of the present invention are also adaptable to other methods of such measurement, as will be readily apparent to those skilled in the art.

Embodiments of the invention that are based on measurements of changes in the transcriptional state in response to a perturbation are particularly preferred. The transcriptional state can be readily measured by techniques of hybridization to arrays of nucleic acid or to arrays of nucleic acid mimic probes, described in the next subsection, or by other gene technologies that are described in subsequent subsections. However measured, the results comprise data values representing RNA abundance ratios, which usually reflect DNA expression ratios (in the absence of differences in RNA degradation rates). Such measurement methods are described in Section 5.4.2, below.

In various alternative embodiments of the invention, other aspects of the biological state such as the translational state, the activity state or mixed aspects can be measured. Details of these alternative embodiments are also described in this section. In particular, such measurement methods are described, below, in Section 5.4.3.

5.4.1. Measurement of Perturbation Response Data

To measure perturbation response data, cells are exposed to a perturbation of interest, such as one of the particular perturbations described in Section 5.5, below. Preferably, the cells are exposed to graded levels of the perturbation of interest, such as exposure to graded levels of a drug or drug candidate. In those embodiments wherein the perturbation is exposure to a compound (e.g., a drug or a drug candidate) the compound is usually added to the nutrient medium of the cells. In the case of yeast, such as S. cerevisiae, it is preferable to harvest the cells in early log phase since expression patterns are relatively insensitive to time of harvest at that time.

The biological state of cells exposed to the perturbation and of cells not exposed to the perturbation are measured according to any of the below described methods. Preferably, transcript or microarrays are used to find the mRNAs with altered expression due to exposure to the perturbation. However, other aspects of the biological state may also be measured to determine, e.g., proteins with altered translation or activity due to exposure to the perturbation. In particularly preferred embodiments, the transcriptional state of cells is measured using two-colored differential hybridization, which is described below. In such embodiments, it is preferable to also measure the transcriptional state with reverse labeling.

5.4.2. Transcriptional State Measurement

In general, measurement of the transcriptional state can be performed using any probe or probes that comprise a polynucleotide sequence and that are immobilized to a solid support or surface. For example, the probes may comprise DNA sequences, RNA sequences or copolymer sequences of DNA and RNA. The polynucleotide sequences of the probes may also comprise DNA and/or RNA analogs or combinations thereof. For example, the polynucleotide sequences of the probe may be full or partial sequences of genomic DNA, cDNA, mRNA or cRNA sequences extracted from cells. The polynucleotide sequences of the probes may also be synthesized nucleotide sequences such as synthetic oligonucleotide sequences. The probe sequences can be synthesized either enzymatically in vivo, enzymatically in vitro (e.g., by PCT) or non-enzymatically in vitro.

In preferred embodiments, the polynucleotide probes are oligonucleotide probes; i.e., the probes comprise oligonucleotide sequences. Oligonucleotide sequences are short sequences of polynucleotides that are preferably between 4 and 200 bases (i.e., nucleotides) in length, and are more preferably between 15 and 150 bases in length. In one embodiment, shorter oligonucleotide sequences are used that are less than 40 bases in length and are preferably between 15 and 30 bases in length. However, a preferred embodiment of the invention uses longer oligonucleotide sequences between 40 and 80 bases in length, with oligonucleotide sequences between 50 and 70 bases in length being preferred, and oligonucleotide sequences between 50 and 60 bases in length being even more preferred.

The probe or probes used in the methods and compositions of the invention are preferably immobilized to a solid support which can be either porous or non-porous. For example, the probes can be polynucleotide sequences that are attached to a nitrocellulose or nylon membrane or filter. Such hybridization probes are well known in the art (see, e.g., Sambrook et al., eds., 1989, Molecular Cloning: A Laboratory Manual, 2nd Ed., Vols. 1-3, Cold Spring Harbor Laboratory, Cold Spring Harbor, N.Y.). Alternatively, the solid support or surface can be a glass or plastic surface or it can be a semi-solid support such as a gel.

Microarrays Generally:

In a particularly preferred embodiment, measurements of the transcriptional state are made by hybridization to microarrays of probes consisting of a solid phase on the surface of which are immobilized a population of polynucleotides, such as a population of DNA or DNA mimics or, alternatively, a population of RNA or RNA mimics. The solid phase may be either porous or non-porous. For example, the probes of the invention may be polynucleotide sequences which are attached to a nitrocellulose or nylon membrane or filter. Alterantively, the solid support or surface can be a glass or plastic surface, or it can be a semi-solid support such as a gel. Microarrays can be employed, e.g., for analyzing the transcriptional state of a cell such as the transcriptional states of cells exposed to graded levels of a drug of interest or to some other perturbation condition.

In preferred embodiments, a microarray comprises a support or surface with ordered array of binding (e.g., hybridizing) sites, e.g., for a plurality of different probes. Microarrays can be made in a number of ways, of which several are described hereinbelow. However produced, microarrays share certain characteristics: The arrays are reproducible, allowing multiple copies of a given array to be produced and easily compared with each other. Preferably, the microarrays are made from materials that are stable under binding (e.g., nucleic acid hybridization) conditions. The microarrays are preferably small, e.g., between 5 cm² and 25 cm², preferably about 12 to 13 cm². However, larger arrays are also contemplated and may be preferable, e.g., for simultaneously evaluating a very large number of different probes.

Preferably, a given binding site or unique set of binding sites in the microarray will specifically bind (e.g., hybridize) to the product of a single gene or gene transcript from a cell or organism (e.g., to a specific mRNA or to a specific cDNA derived therefrom). However, as discussed above, in general other, related or similar sequences will cross hybridize to a given binding site.

The microarrays used in the methods and compositions of the present invention include one or more test probes, each of which has a polynucleotide sequence that is complementary to a subsequence of RNA or DNA to be detected. Each probe preferably has a different nucleic acid sequence, and the position of each probe on the solid surface of the array is preferably known. Indeed, the microarrays are preferably addressable arrays, more preferably positionally addressable arrays. More specifically, each probe of the array is preferably located at a known, predetermined position on the solid support such that the identity (i.e., the sequence) of each probe can be determined from its position on the array (i.e., on the support or surface).

Preferably, the density of probes on a microarray is between about 100 and 1,000 different (i.e., non-identical) probes per 1 cm². More preferably, a microarray of the invention will have between about 1,000 and 5,000 different probes per 1 cm², between about 5,000 and 10,000 different probes per 1 cm², between about 10,000 and 15,000 different probes per 1 cm² or between about 15,000 and 20,000 different probes per 1 cm². In a particularly preferred embodiment, the microarray is a high density array, preferably having a density of between about 1,000 and 5,000 different probes per 1 cm². The microarrays of the invention therefore preferably contain at least 2,500, at least 5,000, at least 10,000, at least 15,000, at least 20,000, at least 25,000, at least 50,000, at least 55,000, at least 100,000 or at least 150,000 different (i.e., non-identical) probes.

In specific embodiments, the density of probes on a microarray is between about 100 and 1,000 different (i.e., non-identical) probes per 1 cm², between 1,000 and 5,000 different probes per 1 cm², between 5,000 and 10,000 different probes per 1 cm², between 10,000 and 15,000 different probes per 1 cm², between 15,000 and 20,000 different probes per 1 cm², between 20,000 and 50,000 different probes per cm², between 50,000 and 100,000 different probes per 1 cm², between 100,000 and 500,000 different probes per 1 cm², or more than 500,000 different (i.e., non-identical) probes per 1 cm².

In one embodiment, the microarray is an array (i.e., a matrix) in which each position represents a discrete binding site for a product encoded by a gene (i.e., for an mRNA or for a cDNA derived therefrom). For example, the binding site can be a DNA or DNA analog to which a particular RNA can specifically hybridize. The DNA or DNA analog can be, e.g., a synthetic oligomer, a fall length cDNA, a less-than full length cDNA, or a gene fragment.

Preferably, the microarrays used in the invention have binding sites (i.e., probes) for one or more genes of interest in the methods of the invention. That is to say, the microarrays preferably have binding sites for one or more genes for which a user wishes to identify one or more functional homologs, e.g., according to the cross-correlation methods of the present invention. The microarrays used in the invention preferably also include microarrays with binding sites for one or more genes that are suspected of being functional homologs of a gene of interest.

A “gene” is typically identified as the portion of DNA that is transcribed by RNA polymerase. Thus, a gene may include a 5′ untranslated region (“UTR”), introns, exons and a 3′ UTR. Thus, a gene comprises at least 25 to 100,000 nucleotides from which a messenger RNA is transcribed in the organism or in some cell in a multicellular organism. The number of genes in a genome can be estimated from the number of mRNAs expressed by the organism, or by extrapolation from a well characterized portion of the genome. When a genome having few introns of an organism of interest, such as yeast, has been sequenced, the number of open reading frames (“ORF”) can be determined and mRNA coding regions identified by analysis of the DNA sequence. For example, the genome of Saccharomyces cerevisiae has been completely sequenced, and is reported to have approximately 6275 ORFs longer than 99 amino acids. Analysis of these ORFs indicates that there are 5885 ORFs that are likely to encode protein products (Goffeau et al., 1996, Science 274:546-567). In contrast, the human genome is estimated to contain approximately 10⁵ genes, although estimates vary from about 35,000 to about 120,000 genes (Crollius et al. (2000) Nat. Genetics 25:235-238; Ewing et al. (2000) Nat. Genetics 25:232-234; Liang et al. (2000) Nat. Genetics 25:239-240).

Preparing Probes for Microarrays:

As noted above, the “probe” to which a particular target polynucleotide molecule specifically hybridizes according to the invention is a complementary polynucleotide sequence to the target polynucleotide. In one embodiment, the probes of the microarray comprises sequences greater than 500 nucleotide bases in length that correspond to a gene or gene fragment. For example, such probes can comprise DNA or DNA “mimics” (e.g., derivatives and analogs) corresponding to at least a portion of one or more genes in an organism's genome. In another embodiment, such probes are complementary RNA or RNA mimics.

DNA mimics are polymers composed of subunits capable of specific, Watson-Crick-like hybridization with DNA, or of specific hybridization with RNA. The DNA mimics can comprise, e.g., nucleic acids modified at the base moiety, at the sugar moiety, or at the phosphate backbone. For example, one particular DNA mimic includes, but is not limited to, phosphorothioates.

Such DNA sequences can be obtained, e.g., by polymerase chain reaction (PCR) amplification of gene segments from, e.g., genomic DNA, mRNA (e.g., from RT-PCR) or from cloned sequences. PCR primers are preferably chosen based on known sequences of the genes or cDNA that result in amplification of unique fragments (i.e., fragments that do not share more than 10 bases of contiguous identical sequence with any other fragment on the microarray). Computer programs that are well known in the art are useful in the design of primers with the required specificity and optimal amplifcation properties, such as Oligo version 5.0 (National Biosciences). Typically, each probe on the microarray will be between 20 bases and 50,000 bases, and usually between 300 bases and 1,000 bases in length. PCR methods are well known in the art and are described, e.g., by Innis et al., eds., 1990, PCR Protocols: A Guide to Methods and Applications, Academic Press, Inc., San Diego, Calif. As will be apparent to one skilled in the art, controlled robotic systems are useful for isolating and amplifying nucleic acids.

An alternative, preferred means for generating the polynucleotide probes for a microarray used in the methods and compositions of the invention is by synthesis of synthetic polynucleotides or oligonucleotides, e.g., using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between 4 and 500 bases in length, more typically between 4 and 200 bases in length, and even more preferably between 15 and 150 bases in length. In embodiments wherein shorter oligonucleotide probes are used, synthetic nucleic acid sequences less than 40 bases in length are preferred, more preferably between 15 and 30 bases in length. In embodiments wherein longer oligonucleotide probes are used, synthetic nucleic acid sequences are preferably between 40 and 80 bases in length, more preferably between 40 and 70 bases in length and even more preferably between 50 and 60 bases in length. In some embodiments, synthetic nucleic acids include non-natural bases, such as, but not limited to, inosine. As noted above, nucleic acid analogs may be used as binding sites for hybridization. An example of a suitable nucleic acid analog is peptide nucleic acid (see, e.g., Egholm et al., 1993, Nature 363:566-568; U.S. Pat. No. 5,539,083).

In other alternative embodiments, the hybridization sites (i.e., the probes) are made from plasmid or phage clones of genes, cDNAs (e.g., expressed sequence tags), or inserts therefrom (see, e.g., Nguyen et al., 1995, Genomics 29:207-209).

Attaching Probes to the Solid Surface:

The probes are preferably attached to a solid support or surface which may be made, e.g., from glass, plastic (e.g., polypropylene, nylon) polyacrylamide, nitrocellulose, a gel, or other porous or nonporous material. A preferred method for attaching the nucleic acids to the surface is by printing on glass plates, as is described generally by Schena et al., 1995, Science 270:467-470. This method is especially useful for preparing microarrays of cDNA (see also DeRisi et al., 1996, Nature Genetics 14:457-460; Shalon et al., 1996, Genome Res. 6:639-645; and Schena et al., 1995, Proc. Natl. Acad. Sci. U.S.A. 93:10539-11286).

Another preferred method for making microarrays is by making high-density oligonucleotide arrays. Techniques are known for producing arrays containing thousand of oligonucleotides complementary to defined sequences and at defined locations on a surface using photolithographic techniques for synthesis in situ (see Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used oligonucleotides (e.g., 25-mers) of known sequence are synthesized directly on a surface such as a derivatized glass slide. Usually, the array produced is redundant with several oligonucleotide molecules per RNA. Oligonucleotide probes can also be chosen to detect particular alternatively spliced mRNAs.

Other methods for making microarrays, e.g., by masking (Maskos and Southern, 1992, Nucl. Acids. Res. 20:1679-1684) can also be used. In principle and as noted above any type of array, for example dot blots on a nylon hybridization membrane (see Sambrook et al., supra) can be used. However, as will be recognized by those skilled in the art, very small arrays will frequently be preferred because hybridization volumes will be smaller.

In a particularly preferred embodiment, micorarrays used in the invention are manufactured by means of an ink jet printing device for oligonucleotide synthesis, e.g., using the methods and systems described by Blanchard in International Patent Publication No. WO 98/41531, published on Sep. 24, 1998; Blanchard et et al., 1996, Biosensors and Bioeletronics 11:687-690; Blanchard, 1998, in Synthetic DNA Arrays in Genetic Engineering, Vol. 20, J. K. Setlow, ed., Plenum Press, New York at pages 111-123. Specifically, the oligonucleotide probes in such microarrays are preferably synthesized by serially depositing individual nucleotides for each probe sequence in an array of “microdroplets” of a high tension solvent such a propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes).

Target Polynucleotide Molecules:

Target polynucleotides which may be analyzed by the methods and compositions of the invention include RNA molecules such as, but by no means limited to, messenger RNA (mRNA) molecules, ribosomal RNA (rRNA) molecules, cRNA molecules (i.e., RNA molecules prepared from cDNA molecules that are transcribed in vivo) and fragments thereof. Target polynucleotides which may also be analyzed by the methods and compositions of the present invention include, but are not limited to DNA molecules such as genomic DNA molecules, cDNA molecules, and fragments thereof including oligonucleotides, ESTs, STSs, etc.

The target polynucleotides may be from any source. For example, the target polynucleotide molecules may be naturally occurring nucleic acid molecules such as genomic or extragenomic DNA molecules isolated from an organism, or RNA molecules, such as mRNA molecules, isolated from an organism. Alternatively, the polynucleotide molecules may be synthesized, including, e.g., nucleic acid molecules synthesized enzymatically in vivo or in vitro, such as cDNA molecules, or polynucleotide molecules synthesized by PCR, RNA molecules synthesized by in vitro transcription, etc. The sample of target polynucleotides can comprise, e.g., molecules of DNA, RNA, or copolymers of DNA and RNA. In preferred embodiments, the target polynucleotides of the invention will correspond to particular genes or to particular gene transcripts (e.g., to particular mRNA sequences expressed in cells or to particular cDNA sequences derived from such mRNA sequences). However, in many embodiments, particularly those embodiments wherein the polynucleotide molecules are derived from mammalian cells, the target polynucleotides may correspond to particular fragments of a gene transcript. For example, the target polynucleotides may correspond to different exons of the same gene, e.g., so that different splice variants of that gene may be detected and/or analyzed.

In preferred embodiments, the target polynucleotides to be analyzed are prepared in vitro from nucleic acids extracted from cells. For example, in one embodiment, RNA is extracted from cells (e.g., total cellular RNA, poly(A)⁺ messenger RNA, fraction thereof) and messenger RNA is purified from the total extracted RNA. Methods for preparing total and poly(A)⁺ RNA are well known in the art, and are described generally, e.g., in Sambrook et al., supra. In one embodiment, RNA is extracted from cells of the various types of interest in this invention using guanidinium thiocyanate lysis followed by CsCl centrifugation (Chirgwin et al., 1979, Biochemistry 18:5294-5299). cDNA is then synthesized from the purified mRNA using, e.g., oligo-dT or random primers. In another preferred embodiment, the target polynucleotides are cRNA prepared from purified messenger RNA extracted from cells. As used herein, cRNA is defined as RNA complementary to the source RNA. The extracted RNAs are amplified using a process in which doubled-stranded cDNAs are synthesized from the RNAs using a primer linked to an RNA polymerase promoter in a direction capable of directing transcription of anti-sense RNA. Anti-sense RNAs or cRNAs are then transcribed from the second strand of the double-stranded cDNAs using an RNA polymerase (see, e.g., U.S. Pat. Nos. 5,891,636, 5,716,785; 5,545,522 and 6,132,997; see also, U.S. patent application Ser. No. 09/411,074, filed Oct. 4, 1999 by Linsley and Schelter, and U.S. Provisional Patent Application Serial No. to be assigned, Attorney Docket No. 9301-124-888, filed on Nov. 28, 2000, by Ziman et al.). Both oligo-dT primers (U.S. Pat. Nos. 5,545,522 and 6,132,997) or random primers (U.S. Provisional Patent Application, Serial No. to be assigned, Attorney Docket No. 9301-124-888, filed Nov. 28, 2000, by Ziman et al.) that contain an RNA polymerase promoter or complement thereof can be used. Preferably, the target polynucleotides are short and/or fragmented polynucleotide molecules that are representative of the original nucleic acid population of the cell.

The target polynucleotides to be analyzed by the methods and compositions of the invention are preferably detectably labeled. For example, cDNA can be labeled directly, e.g., with nucleotide analogs, or indirectly, e.g., by making a second, labeled cDNA strand using the first strand as a template. Alternatively, the double-stranded cDNA can be transcribed into cRNA and labeled.

Preferably, the detectable label is a fluorescent label, e.g., by incorporation of nucleotide analogs. Other labels suitable for use in the present invention include, but are not limited to, biotin, imminobiotin, antigens, cofactors, dinitrophenol, lipoic acid, olefinic compounds, detectable polypeptides, electron rich molecules, enzymes capable of generating a detectable signal by action upon a substrate, and radioactive isotopes. Preferred radioactive isotopes include ³²P, ³⁵S, ¹⁴C, ¹⁵N and ¹²⁵I. Fluorescent molecules suitable for the present invention include, but are not limited to, fluorescein and its derivatives, rhodamine and its derivatives, texas red, 5′carboxy-fluorescein (“FMA”), 2′,7′-dimethoxy-4′,5′-dichloro-6-carboxy-fluorescein (“JOE”), N,N,N′,N′-tetramethyl-6-carboxy-rhodamine (“TAMRA”), 6′carboxy-X-rhodamine (“ROX”), HEX, TET, IRD40, and IRD41. Fluroescent molecules that are suitable for the invention further include: cyamine dyes, including by not limited to Cy3, Cy3.5 and Cy5; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes which will be known to those who are skilled in the art. Electron rich indicator molecules suitable for the present invention include, but are not limited to, ferritin, hemocyanin, and colloidal gold. Alternatively, in less preferred embodiments the target polynucleotides may be labeled by specifically complexing a first group to the polynucleotide. A second group, covalently linked to an indicator molecules and which has an affinity for the first group, can be used to indirectly detect the target polynucleotide. In such an embodiment, compounds suitable for use as a first group include, but are not limited to, biotin and iminobiotin. Compounds suitable for use as a second group include, but are not limited to, avidin and streptavidin.

Hybridization to Microarrays:

Nucleic acid hybridization and wash conditions are chosen so that the polynucleotide molecules to be analyzed by the invention (referred to herein as the “target polynucleotide molecules) specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. General parameters for specific (i.e., stringent) hybridization conditions for nucleic acids are described in Sambrook et al., (supra), and in Ausubel et al., 1987, Current Protocols in Molecular Biology, Greene Publishing and Wiley-Interscience, New York. When the cDNA microarrays of Schena et al. are used, typical hybridization conditions are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Shena et al., 1996, Proc. Natl. Acad. Sci. U.S.A. 93:10614). Useful hybridization conditions are also provided in, e.g., Tijessen, 1993, Hybridization With Nucleic Acid Probes, Elsevier Science Publishers B. V. and Kricka, 1992, Nonisotopic DNA Probe Techniques, Academic Press, San Diego, Calif.

Particularly preferred hybridization conditions for use with the screening and/or signaling chips of the present invention include hybridization at a temperature at or near the mean melting temperature of the probes (e.g., within 5° C., more preferably within 2° C.) in 1 M NaCl, 50 mM MES buffer (pH 6.5), 0.5% sodium sarcosine and 30% formamide.

Signal Detection and Data Analysis:

It will be appreciated that when cDNA or cRNA complementary to the RNA of a cell is made and hybridized to a microarray under suitable hybridization conditions, the level of hybridization to the site in the array corresponding to any particular gene will reflect the prevalence in the cell of mRNA transcribed from that gene. For example, when detectably labeled (e.g., with a fluorophore) cDNA or cRNA complementary to the total cellular mRNA is hybridized to a microarray, the site on the array corresponding to a gene (i.e., capable of specifically binding the product of the gene) that is not transcribed in the cell will have little or no signal (e.g., fluorescent signal), and a gene for which the encoded mRNA is prevalent will have a relatively strong signal.

In preferred embodiments, cDNAs or cRNAs from two different cells are hybridized to the binding sites of the microarray. In the case of the instant invention, one cell is a wild-type cell and another cell of the same type has a mutation in a specific gene. The cDNA or cRNA derived from each of the two cell types are differently labeled so that they can be distinguished. In one embodiment, for example, cDNA or cRNA from a cell with a mutation in a specific gene is synthesized using a fluorescein-labeled dNTP, and cDNA or cRNA from a second, wild-type cell is synthesized using a rhodamine-labeled dNTP. When the two cDNAs or cRNAs are mixed and hybridized to the microarray, the relative intensity of signal from each cDNA or cRNA set is determined for each site on the array, and any relative difference in abundance of a particular mRNA is thereby detected.

In the example described above, the cDNA or cRNA from the mutant cell will fluoresce green when the fluorophore is stimulated, and the cDNA or cRNA from the wild-type cell will fluoresce red. As a result, when the mutation has no effect, either directly or indirectly, on the relative abundance of a particular mRNA in a cell, the mRNA will be equally prevalent in both cells, and, upon reverse transcription, red-labeled and green-labeled cDNA or cRNA will be equally prevalent. When hybridized to the microarray, the binding site(s) for that species of RNA will emit wavelength characteristic of both fluorophores. In contrast, when the either directly or indirectly increases the prevalence of the mRNA in the cell, the ratio of green to red fluorescence will increase. When the mutation decreases the mRNA prevalence, the ratio will decrease.

In preferred embodiments, cDNAs or cRNAs from cell samples from two different conditions are hybridized to the binding sites of the microarray using a two-color protocol. In the case of drug responses one cell sample is exposed to a drug and another cell sample of the same type is not exposed to the drug. In the case of overexpression of one or more genes, one cell has a variation in gene dosage and the other has a wild-type gene dosage. The cDNA or cRNA derived from each of the two cell types are differently labeled (e.g., with Cy3 and Cy5) so that they can be distinguished. In one embodiment, for example, cDNA or cRNA from a cell treated with a drug is synthesized using a fluorescein-labeled dNTP, and cDNA or cRNA from a second, untreated cell is synthesized using a rhodamine-labeled dNTP. When the two cDNAs or cRNAs are mixed and hybridized to the microarray, the relative signal intensity from each cDNA or cRNA set is determined for each site on the array, and any relative difference in abundance of a particular gene is detected.

In the example described above, the cDNA or cRNA from the drug-treated cell will fluoresce green when the fluorophore is stimulated and the cDNA or cRNA from the untreated cell will fluoresce red. As a result, when the drug treatment has no effect, either directly or indirectly, on transcription, the expression patterns will be indistinguishable in both cells and, upon reverse transcription, red-labeled and green-labeled cDNA or cRNA will be equally prevalent. When hybridized to the microarray, the binding site(s) for that species of RNA will emit wavelengths characteristic of both fluorophores. In contrast, when the drug-exposed cell is treated with a drug that, directly or indirectly, changes the transcription of a particular gene in the cell, the expression profile as represented by ratio of green to red fluorescence for each binding site on the array will change. When the drug increases the prevalence of an mRNA, the ratio for each expressed gene will increase, whereas when the drug decreases the prevalence of an mRNA, the ratio for each expressed gene will decrease.

The use of a two-color fluorescence labeling and detection scheme to define alterations in gene expression has been described, e.g., in Shena et al., 1995, Science 270:467-470. An advantage of using cDNA or cRNA labeled with two different fluorophores is that a direct and internally controlled comparison of the mRNA levels corresponding to each arrayed gene in two cell genotypes can be made, and variations due to minor differences in experimental conditions (e.g., hybridization conditions) will not affect subsequent analyses.

In a preferred embodiment, the fluorescent labels in two-color differential hybridization experiments are reversed to reduce biases peculiar to individual genes or array spot locations, and consequently, to reduce experimental error. In other words, it is preferable to first measure gene expression with one labeling (e.g., labeling wild-type cells with a first fluorophore and mutant cells with a second fluorophore) of the mRNA from the two cells being measured, and then to measure gene expression from the two cells with reversed labeling (e.g., labeling wild-type cells with the second fluorophre and mutant cells with the first fluorophore).

When fluorescently labeled probes are used, the fluorescence emissions at each site of a transcript array can be, preferably, detected by scanning confocal laser microscopy or a charge-coupled device (“CCD”). In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Res. 6:639-645). In a preferred embodiment, the arrays are scanned with a laser fluorescent scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al., 1996, Genome Res. 6:639-645. Alternatively, the fiber-optic bundle described by Ferguson et al., 1996, Nature Biotech. 14:1681-1684, may be used to monitor mRNA abundance levels at a large number of sites simultaneously.

Signals are recorded and, in a preferred embodiment, analyzed by computer, e.g., using a 12 bit analog to digital board. In one embodiment, the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors may be made. For any particular hybridization site on the transcript array, a ratio of the emission of the two fluorophores can be calculated. The ratio is independent of the absolute expression level of the cognate gene, but is useful for genes whose expression is significantly modulated by alterations in the genotype of a cell.

According to the method of the invention, if a gene's expression is affected, it is scored as a perturbation and its magnitude determined (i.e., the abundance is different in the two sources of mRNA tested) or as not perturbed (i.e., the relative abundance is the same). As used herein, any difference between the two sources of RNA that can be reliably measured may be used to score a perturbation. Present detection methods allow for reliable detection of a difference of an order of about 3-fold to about 5-fold. Accordingly, in various embodiments of the present invention, a factor of about 2 (i.e., RNA is twice as abundant in one source as it is in the other source), 3 (three times as abundant), or 5 (five times as abundant), is scored as a perturbation. It is widely expected that more sensitive methods for the detection of differences in RNA levels will be developed. Accordingly, when such methods become available, the present invention can be practiced with smaller differences between the two sources of RNA. For example, in some embodiments, a factor of about 25% or more will be used to score a perturbation. In yet another embodiment, a difference of about 50% or more between the two sources of RNA will be used to score a perturbation.

Preferably, in addition to identifying the effect of a perturbation as positive or negative, it is advantageous to determine the magnitude of the effect of the perturbation. This can be carried out, as noted above, by calculating the ratio of the emission of the two fluorophores used for differential labeling, or by analogous methods that will be readily apparent to those of skill in the art.

Other Methods of Transcriptional State Measurement:

The transcriptional state of a cell may be measured by other gene expression technologies known in the art. Several such technologies produce pools of restriction fragments of limited complexity for electrophoretic analysis, such as methods combining double restriction enzyme digestion with phasing primers (see, e.g., European Patent O 534858 A1 filed Sep. 24, 1992 by Zabeau et al.) or methods selecting restriction fragments with sites closest to a defined mRNA end (see, e.g., Prashar et al., 1996, Proc. Natl. Acad. Sci. U.S.A. 93:659-663). Other methods statistically sample cDNA pools, such as by sequencing sufficient bases (e.g., 20-50 bases) in each of multiple cDNAs to identify each cDNA, or by sequencing short tags (e.g., 9-10 bases) which are generated at known positions relative to a defined mRNA end (see, e.g., Velculescu, 1995, Science 270:484-487).

Such methods and systems of measuring transcriptional state, although less preferable than microarrays, may nevertheless be used in the present invention.

5.4.3. Measurements of Other Aspects of Biological State

As will be apparent to those skilled in the art, the methods of the present invention are equally applicable to measurements of other cellular constituents and aspects of the biological state besides the transcription state (i.e., besides measurements of mRNA levels). For example, in various embodiments of the invention, aspects of the biological state such as the translational state, the activity state, or mixed aspects thereof can be measured in order to obtain perturbation response profiles for the invention. Details of such embodiments are described in this section.

Translational State Measurement:

Measurements of the translational state may be performed according to any of several methods that are known in the art. For example, whole genome monitoring of protein (i.e., the “proteome;” see, e.g. Goffea et al., supra) can be carried out by constructing a microarray in which binding sites comprise immobilized, preferably monoclonal, antibodies specific to a plurality of protein species encoded by the cell genome. Preferably, antibodies are present for a substantial fraction of the encoded proteins or at least for those proteins for which functional homologs are to be identified (e.g., by the cross-correlation methods of the present invention) and/or for proteins that are suspected of being functional homologs of a particular protein of interest. Methods for making monoclonal antibodies are well known in the art (see, e.g., Harlow and Lane, 1988, Antibodies: A Laboratory Manual, Cold Spring Harbor, N.Y.). In a preferred embodiment, monoclonal antibodies are raised against synthetic peptide fragments designed based on the genomic sequence of the cell. With such an antibody array, proteins from the cell are contacted to the array and their binding is assayed with assays known in the art.

Alternatively, proteins can be separated by two-dimensional gel electrophoresis systems. Two-dimensional gel electrophoresis is well known in the art and typically involves iso-electric focusing along a first dimension followd by SDS-PAGE electrophoresis along a second dimension. See, e.g., Hames et al., 1990, Gel Electrophoresis of Proteins: A Practical Approach, IRL Press, New York; Shevchenko et al, 1996, Proc. Natl. Acad. Sci. U.S.A. 93:1440-1445; Sagliocco et al., 1996, Yeast 12:1519-1533; and Lander, 1996, Science 274:536-539. The resulting electropherograms can be analyzed by numerous techniques, including mass spectrometric techniques, western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug or in cells modified by, e.g., deletion or over-expression of a specific gene.

Activity State Measurements:

Where activities of proteins relevant to the characterization of drug action can be measured, embodiments of this invention can be based on such measurements. Activity measurements can be performed by any functional, biochemical or physical means appropriate to the particular activity being characterized. Where the activity involves a chemical transformation, the cellular protein can be contacted with the natural substrate(s) and the rate of transformation measured. Where the activity involves association in multimeric units, for example association of an activated DNA binding commplex with DNA, the amount of associated protein or secondary consequences of the association, such as amounts of mRNA transcribed, can be measured. Also, where only a functional activity is known, for example as in cell cycle control, performance of the function can be observed. However known or measured, the changes in protein activities form the response data analyzed by the foregoing methods of this invention.

Mixed Aspects of Biological State:

In alternative and non-limiting embodiments, response data may be formed of mixed aspects of the biological state of a cell. Response data can be constructed from combinations of, e.g., changes in certain mRNA abundances, changes in certain protein abundances and changes in certain protein activities.

5.5. Targeted Perturbation Methods

Methods for targeted perturbation of biological pathways at various levels of a cell are increasingly widely known and applied in the art. Any such methods that are capable of specifically targeting and controllably modifying (e.g., either by a graded increase or activation or by a graded decrease or inhibition) specific cellular constituents (e.g., gene expression, RNA concentrations, protein abundances, protein activities, or so forth) can be employed in performing pathway perturbations. Controllable modifications of cellular constituents consequentially controllably perturb pathways originating at the modified cellular constituents. Such pathways originating at specific cellular constituents are preferably employed to represent drug action in this invention. Preferable modification methods are capable of individually targeting each of a plurality of cellular constituents and most preferably a substantial fraction of such cellular constituents.

The following methods are exemplary of those that can be used to modify cellular constituents and thereby to produce pathway perturbations which generate the pathway responses used in the steps of the methods of this invention as previously described. This invention is adaptable to other methods for making controllable perturbations to pathways, and especially to cellular constituents from which pathways originate.

Pathway perturbations are preferably made in cells of cell types derived from any organism for which genomic or expressed sequence information is available and for which methods are available that permit controllably modification of the expression of specific genes. Genome sequencing is currently underway for several eukaryotic organisms, including humans, nematodes, Arabidopsis, and flies. In a preferred embodiment, the invention is carried out using a yeast, with Saccharomyces cerevisiae most preferred because the sequence of the entire genome of a S. cerevisiae strain has been determined. In addition, well-established methods are available for controllably modifying expression of yeast genes. A preferred strain of yeast is a S. cerevisiae strain for which yeast genomic sequence is known, such as strain S288C or substantially isogeneic derivatives of it (see, e.g., Dujon et al., 1994, Nature 369:371-378; Bussey et al., 1995, Proc. Natl. Acad. Sci. U.S.A. 92:3809-3813; Feldmann et al., 1994, E.M.B.O. J. 13:5795-5809; Johnston et al., 1994, Science 265:2077-2082; Galibert et al, 1996, E.M.B.O. J. 15:2031-2049). However, other strains may be used as well. Yeast strains are available, e.g., from American Type Culture Collection, 10801 University Boulevard, Manassas, Va. 20110-2209. Standard techniques for manipulating yeast are described in C. Kaiser, S. Michaelis, & A. Mitchell, 1994, Methods in Yeast Genetics: A Cold Spring Harbor Laboratory Course Manual, Cold Spring Harbor Laboratory Press, New York; and Sherman et al., 1986, Methods in Yeast Genetics: A Laboratory Manual, Cold Spring Harbor Laboratory, Cold Spring Harbor. N.Y.

The exemplary methods described in the following include use of titratable expression systems, use of transfection or viral transduction systems, direct modifications to RNA abundances or activities, direct modifications of protein abundances, and direct modification of protein activities including use of drugs (or chemical moieties in general) with specific known action.

5.5.1. Titratable Expression Systems

Any of the several known titratable, or equivalently controllable, expression systems available for use in the budding yeast Saccharomyces cerevisiae are adaptable to this invention (Mumberg et al., 1994, Nucl. Acids Res. 22:5767-5768). Usually, gene expression is controlled by transcriptional controls, with the promoter of the gene to be controlled replaced on its chromosome by a controllable, exogenous promoter. The most commonly used controllable promoter in yeast is the GAL1 promoter (Johnston et al., 1984, Mol Cell. Biol. 8:1440-1448). The GAL1 promoter is strongly repressed by the presence of glucose in the growth medium, and is gradually switched on in a graded manner to high levels of expression by the decreasing abundance of glucose and the presence of galactose. The GAL1 promoter usually allows a 5-100 fold range of expression control on a gene of interest.

Other frequently used promoter systems include the MET25 promoter (Kerjan et al., 1986, Nucl. Acids. Res. 14:7861-7871), which is induced by the absence of methionine in the growth medium, and the CUP1 promoter, which is induced by copper (Mascorro-Gallardo et al., 1996, Gene 172:169-170). All of these promoter systems are controllable in that gene expression can be incrementally controlled by incremental changes in the abundances of a controlling moiety in the growth medium.

One disadvantage of the above listed expression systems is that control of promoter activity (effected by, e.g., changes in carbon source, removal of certain amino acids), often causes other changes in cellular physiology which independently alter the expression levels of other genes. A recently developed system for yeast, the Tet system, alleviates this problem to a large extent (Gari et al., 1997, Yeast 13:837-848). The Tet promoter, adopted from mammalian expression systems (Gossen et al., 1995, Proc. Nat. Acad. Sci. USA 89:5547-5551) is modulated by the concentration of the antibiotic tetracycline or the structurally related compound doxycycline. Thus, in the absence of doxycycline, the promoter induces a high level of expression, and the addition of increasing levels of doxycycline causes increased repression of promoter activity. Intermediate levels gene expression can be achieved in the steady state by addition of intermediate levels of drug. Furthermore, levels of doxycycline that give maximal repression of promoter activity (10 micrograms/ml) have no significant effect on the growth rate on wild type yeast cells (Gari et al., 1997, Yeast 13:837-848).

In mammalian cells, several means of titrating expression of genes are available (Spencer, 1996, Trends Genet. 12:181-187). As mentioned above, the Tet system is widely used, both in its original form, the “forward” system, in which addition of doxycycline represses transcription, and in the newer “reverse” system, in which doxycycline addition stimulates transcription (Gossen et al., 1995, Proc. Natl. Acad. Sci. USA 89:5547-5551; Hoffmann et al., 1997, Nucl. Acids. Res. 25:1078-1079; Hofmann et al., 1996, Proc. Natl. Acad. Sci. USA 83:5185-5190; Paulus et al., 1996, Journal of Virology 70:62-67). Another commonly used controllable promoter system in mammalian cells is the ecdysone-inducible system developed by Evans and colleagues (No et al., 1996, Proc. Nat. Acad. Sci. USA 93:3346-3351), where expression is controlled by the level of muristerone added to the cultured cells. Finally, expression can be modulated using the “chemical-induced dimerization” (CID) system developed by Schreiber, Crabtree, and colleagues (Belshaw et al., 1996, Proc. Nat. Acad. Sci. USA 93:4604-4607; Spencer, 1996, Trends Genet. 12:181-187) and similar systems in yeast. In this system, the gene of interest is put under the control of the CID-responsive promoter, and transfected into cells expressing two different hybrid proteins, one comprised of a DNA-binding domain fused to FKBP12, which binds FK506. The other hybrid protein contains a transcriptional activation domain also fused to FKBP12. The CID inducing molecule is FK1012, a homodimeric version of FK506 that is able to bind simultaneously both the DNA binding and transcriptional activating hybrid proteins. In the graded presence of FK1012, graded transcription of the controlled gene is activated.

For each of the mammalian expression systems described above, as is widely known to those of skill in the art, the gene of interest is put under the control of the controllable promoter, and a plasmid harboring this construct along with an antibiotic resistance gene is transfected into cultured mammalian cells. In general, the plasmid DNA integrates into the genome, and drug resistant colonies are selected and screened for appropriate expression of the regulated gene. Alternatively, the regulated gene can be inserted into an episomal plasmid such as pCEP4 (Invitrogen, Inc.), which contains components of the Epstein-Barr virus necessary for plasmid replication.

In a preferred embodiment, titratable expression systems, such as the ones described above, are introduced for use into cells or organisms lacking the corresponding endogenous gene and/or gene activity, e.g., organisms in which the endogenous gene has been disrupted or deleted. Methods for producing such “knock outs” are well known to those of skill in the art, see e.g., Pettitt et al., 1996, Development 122:4149-4157; Spradling et al., 1995, Proc. Natl. Acad. Sci. USA, 92:10824-10830; Ramirez-Solis et al., 1993, Methods Enzymol. 225:855-878; and Thomas et al., 1987, Cell 51:503-512.

5.5.2. Transfection Systems for Mammalian Cells

Transfection or viral transduction of target genes can introduce controllable perturbations in biological pathways in mammalian cells. Preferably, transfection or transduction of a target gene can be used with cells that do not naturally express the target gene of interest. Such non-expressing cells can be derived from a tissue not normally expressing the target gene or the target gene can be specifically mutated in the cell. The target gene of interest can be cloned into one of many mammalian expression plasmids, for example, the pcDNA3.1 +/− system (Invitrogen, Inc.) or retroviral vectors, and introduced into the non-expressing host cells. Transfected or transduced cells expressing the target gene may be isolated by selection for a drug resistance marker encoded by the expression vector. The level of gene transcription is monotonically related to the transfection dosage. In this way, the effects of varying levels of the target gene may be investigated.

A particular example of the use of this method is the search for drugs that target the src-family protein tyrosine kinase, lck, a key component of the T cell receptor activation pathway (Anderson et al., 1994, Adv. Immunol. 56:171-178). Inhibitors of this enzyme are of interest as potential immunosuppressive drugs (Hanke J H, 1996, J. Biol. Chem 271(2):695-701). A specific mutant of the Jurkat T cell line (JcaM1) is available that does not express lck kinase (Straus et al., 1992, Cell 70:585-593). Therefore, introduction of the lck gene into JCaM1 by transfection or transduction permits specific perturbation of pathways of T cell activation regulated by the lck kinase. The efficiency of transfection or transduction, and thus the level of perturbation, is dose related. The method is generally useful for providing perturbations of gene expression or protein abundances in cells not normally expressing the genes to be perturbed.

5.5.3. Methods of Modifying RNA Abundances or Activities

Methods of modifying RNA abundances and activities currently fall within three classes, ribozymes, antisense species, and RNA aptamers (Good et al., 1997, Gene Therapy 4: 45-54). Controllable application or exposure of a cell to these entities permits controllable perturbation of RNA abundances.

Ribozymes are RNAs which are capable of catalyzing RNA cleavage reactions. (Cech, 1987, Science 236:1532-1539; PCT International Publication WO 90/11364, published Oct. 4, 1990; Sarver et al., 1990, Science 247: 1222-1225). “Hairpin” and “hammerhead” RNA ribozymes can be designed to specifically cleave a particular target mRNA. Rules have been established for the design of short RNA molecules with ribozyme activity, which are capable of cleaving other RNA molecules in a highly sequence specific way and can be targeted to virtually all kinds of RNA. (Haseloff et al., 1988, Nature 334:585-591; Koizumi et al., 1988, FEBS Lett. 228:228-230; Koizumi et al., 1988, FEBS Lett. 239:285-288). Ribozyme methods involve exposing a cell to, inducing expression in a cell, etc. of such small RNA ribozyme molecules. (Grassi and Marini, 1996, Annals of Medicine 28: 499-510; Gibson, 1996, Cancer and Metastasis Reviews 15: 287-299).

Ribozymes can be routinely expressed in vivo in sufficient number to be catalytically effective in cleaving mRNA, and thereby modifying mRNA abundances in a cell. (Cotten et al., 1989, EMBO J. 8:3861-3866). In particular, a ribozyme coding DNA sequence, designed according to the previous rules and synthesized, for example, by standard phosphoramidite chemistry, can be ligated into a restriction enzyme site in the anticodon stem and loop of a gene encoding a tRNA, which can then be transformed into and expressed in a cell of interest by methods routine in the art. Preferably, an inducible promoter (e.g., a glucocorticoid or a tetracycline response element) is also introduced into this construct so that ribozyme expression can be selectively controlled. tDNA genes (i.e., genes encoding tRNAs) are useful in this application because of their small size, high rate of transcription, and ubiquitous expression in different kinds of tissues. Therefore, ribozymes can be routinely designed to cleave virtually any mRNA sequence, and a cell can be routinely transformed with DNA coding for such ribozyme sequences such that a controllable and catalytically effective amount of the ribozyme is expressed. Accordingly the abundance of virtually any RNA species in a cell can be perturbed.

In another embodiment, activity of a target RNA (preferable mRNA) species, specifically its rate of translation, can be controllably inhibited by the controllable application of antisense nucleic acids. An “antisense” nucleic acid as used herein refers to a nucleic acid capable of hybridizing to a sequence-specific (e.g., non-poly A) portion of the target RNA, for example its translation initiation region, by virtue of some sequence complementarity to a coding and/or non-coding region. The antisense nucleic acids of the invention can be oligonucleotides that are double-stranded or single-stranded, RNA or DNA or a modification or derivative thereof, which can be directly administered in a controllable manner to a cell or which can be produced intracellularly by transcription of exogenous, introduced sequences in controllable quantities sufficient to perturb translation of the target RNA.

Preferably, antisense nucleic acids are of at least six nucleotides and are preferably oligonucleotides (ranging from 6 to about 200 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide may include other appending groups such as peptides, or agents facilitating transport across the cell membrane (see, e.g., Letsinger et al, 1989, Proc. Natl. Acad. Sci. U.S.A. 86: 6553-6556; Lemaitre et al., 1987, Proc. Natl. Acad. Sci. U.S.A. 84: 648-652; PCT Publication No. WO 88/09810, published Dec. 15, 1988), hybridization-triggered cleavage agents (see, e.g., Krol et et al., 1988, BioTechniques 6: 958-976) or intercalating agents (see, e.g., Zon, 1988, Pharm. Res. 5: 539-549).

In a preferred aspect of the invention, an antisense oligonucleotide is provided, ii preferably as single-stranded DNA. The oligonucleotide may be modified at any position on its structure with constituents generally known in the art.

The antisense oligonucleotides may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the oligonucleotide is a 2-α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15: 6625-6641).

The oligonucleotide may be conjugated to another molecule, e.g., a peptide, hybridization triggered cross-linking agent, transport agent, hybridization-triggered cleavage agent, etc.

The antisense nucleic acids of the invention comprise a sequence complementary to at least a portion of a target RNA species. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a target RNA it may contain and still form a stable duplex (or triplex, as the case may be). One skilled in the art can ascertain a tolerable degree of mismatch by use of standard procedures to determine the melting point of the hybridized complex. The amount of antisense nucleic acid that will be effective in the inhibiting translation of the target RNA can be determined by standard assay techniques.

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16: 3209), methylphosphonate oligonucleotides can be prepared by use of controlled pore glass polymer supports (Sarin et al, 1988, Proc. Natl. Acad. Sci. U.S.A. 85: 7448-7451), etc. In another embodiment, the oligonucleotide is a 2′-0-methylribonucleotide (Inoue et al., 1987, Nucl. Acids Res. 15: 6131-6148), or a chimeric RNA-DNA analog (Inoue et al., 1987, FEBS Lett. 215: 327-330).

The synthesized antisense oligonucleotides can then be administered to a cell in a controlled manner. For example, the antisense oligonucleotides can be placed in the growth environment of the cell at controlled levels where they may be taken up by the cell. The uptake of the antisense oligonucleotides can be assisted by use of methods well known in the art.

In an alternative embodiment, the antisense nucleic acids of the invention are controllably expressed intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding the antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequences encoding the antisense RNAs can be by any promoter known in the art to act in a cell of interest. Such promoters can be inducible or constitutive. Most preferably, promoters are controllable or inducible by the administration of an exogenous moiety in order to achieve controlled expression of the antisense oligonucleotide. Such controllable promoters include the Tet promoter. Less preferably usable promoters for mammalian cells include, but are not limited to: the SV40 early promoter region (Bernoist and Chambon, 1981, Nature 290: 304-310), the promoter contained in the 3′ long terminal repeat of Rous sarcoma virus (Yamamoto et al., 1980, Cell 22: 787-797), the herpes thymidine kinase promoter (Wagner et al., 1981, Proc. Natl. Acad. Sci. U.S.A. 78: 1441-1445), the regulatory sequences of the metallothionein gene (Brinster et al., 1982, Nature 296: 39-42), etc.

Therefore, antisense nucleic acids can be routinely designed to target virtually any mRNA sequence, and a cell can be routinely transformed with or exposed to nucleic acids coding for such antisense sequences such that an effective and controllable amount of the antisense nucleic acid is expressed. Accordingly the translation of virtually any RNA species in a cell can be controllably perturbed.

In a further embodiment, RNA aptamers can be introduced into or expressed in a cell. RNA aptamers are specific RNA ligands for proteins, such as for Tat and Rev RNA (Good et al, 1997, Gene Therapy 4: 45-54) that can specifically inhibit their translation.

Post-transcriptional gene silencing (PTGS) or RNA interference (RNAi) can also be used to modify RNA abundances (Guo et al., 1995, Cell 81:611-620; Fire et al., 1998, Nature 391:806-811). In RNAi, dsRNAs are injected into cells to specifically block expression of its homologous gene. In particular, in RNAi, both the sense strand and the anti-sense strand can inactivate the corresponding gene. It is suggested that the dsRNAs are cut by nucleases into 21-23 nucleotide fragments. These fragments hybridize to the homologous region of their corresponding mRNAs to form double-stranded segments, which are then degraded by nucleases (Grant, 1999, Cell 96:303-306; Zamore et al., 2000, Cell 101:25-33; Bass, 2000, Cell 101:235-238; Petcherski et al., 2000, Nature 405:364-368). It has been hypothesized that RNAi may perform in vivo functions of, inter alia, transposon silencing (Tabara et al. (1999) Cell 99:123-32), defending against viruses (Ratcliff et al. (1997) Science 276:1558-1560) and reducing accumulation of RNAs with sequence similarity to nucleic acids that have been introduced into cells (Hamilton et al., 1999, Science 286:950-952). Therefore, in one embodiment, one or more dsRNAs having sequences homologous to the sequences of one or more mRNAs whose abundances are to be modified are transfected into a cell or tissue sample. Any standard methods for introducing nucleic acids into cells can be used.

5.5.4. Methods of Modifying Protein Abundances

Methods of modifying protein abundances include, inter alia, those altering protein degradation rates and those using antibodies (which bind to proteins affecting abundances of activities of native target protein species). Increasing (or decreasing) the degradation rates of a protein species decreases (or increases) the abundance of that species. Methods for controllably increasing the degradation rate of a target protein in response to elevated temperature and/or exposure to a particular drug, which are known in the art, can be employed in this invention. For example, one such method employs a heat-inducible or drug-inducible N-terminal degron, which is an N-terminal protein fragment that exposes a degradation signal promoting rapid protein degradation at a higher temperature (e.g., 37° C.) and which is hidden to prevent rapid degradation at a lower temperature (e.g., 23° C.) (Dohmen et al., 1994, Science 263:1273-1276). Such an exemplary degron is Arg-DHFR^{ts}, a variant of murine dihydrofolate reductase in which the N-terminal Val is replaced by Arg and the Pro at position 66 is replaced with Leu. According to this method, for example, a gene for a target protein, P, is replaced by standard gene targeting methods known in the art (Lodish et al., 1995, Molecular Biology of the Cell, Chpt. 8, New York: W. H. Freeman and Co.) with a gene coding for the fusion protein Ub-Arg-DHFR^{ts}-P (“Ub” stands for ubiquitin). The N-terminal ubiquitin is rapidly cleaved after translation exposing the N-terminal degron. At lower temperatures, lysines internal to Arg-DHFR^{ts} are not exposed, ubiquitination of the fusion protein does not occur, degradation is slow, and active target protein levels are high. At higher temperatures (in the absence of methotrexate), lysines internal to Arg-DHFR^{ts} are exposed, ubiquitination of the fusion protein occurs, degradation is rapid, and active target protein levels are low. Heat activation of degradation is controllably blocked by exposure methotrexate. This method is adaptable to other N-terminal degrons which are responsive to other inducing factors, such as drugs and temperature changes.

Target protein abundances and also, directly or indirectly, their activities can also be decreased by (neutralizing) antibodies. By providing for controlled exposure to such antibodies, protein abundances/activities can be controllably modified. For example, antibodies to suitable epitopes on protein surfaces may decrease the abundance, and thereby indirectly decrease the activity, of the wild-type active form of a target protein by aggregating active forms into complexes with less or minimal activity as compared to the wild-type unaggregated wild-type form. Alternately, antibodies may directly decrease protein activity by, e.g., interacting directly with active sites or by blocking access of substrates to active sites. Conversely, in certain cases, (activating) antibodies may also interact with proteins and their active sites to increase resulting activity. In either case, antibodies (of the various types to be described) can be raised against specific protein species (by the methods to be described) and their effects screened. The effects of the antibodies can be assayed and suitable antibodies selected that raise or lower the target protein species concentration and/or activity. Such assays involve introducing antibodies into a cell (see below), and assaying the concentration of the wild-type amount or activities of the target protein by standard means (such as immunoassays) known in the art. The net activity of the wild-type form can be assayed by assay means appropriate to the known activity of the target protein.

Antibodies can be introduced into cells in numerous fashions, including, for example, microinjection of antibodies into a cell (Morgan et al., 1988, Immunology Today 9:84-86) or transforming hybridoma mRNA encoding a desired antibody into a cell (Burke et al., 1984, Cell 36:847-858). In a further technique, recombinant antibodies can be engineering and ectopically expressed in a wide variety of non-lymphoid cell types to bind to target proteins as well as to block target protein activities (Biocca et al., 1995, Trends in Cell Biology 5:248-252). Preferably, expression of the antibody is under control of a controllable promoter, such as the Tet promoter. A first step is the selection of a particular monoclonal antibody with appropriate specificity to the target protein (see below). Then sequences encoding the variable regions of the selected antibody can be cloned into various engineered antibody formats, including, for example, whole antibody, Fab fragments, Fv fragments, single chain Fv fragments (V_{H} and V_{L} regions united by a peptide linker) (“ScFv” fragments), diabodies (two associated ScFv fragments with different specificities), and so forth (Hayden et al., 1997, Current Opinion in Immunology 9:210-212). Intracellularly expressed antibodies of the various formats can be targeted into cellular compartments (e.g., the cytoplasm, the nucleus, the mitochondria, etc.) by expressing them as fusions with the various known intracellular leader sequences (Bradbury et al, 1995, Antibody Engineering, vol. 2, Borrebaeck ed., IRL Press, pp 295-361). In particular, the ScFv format appears to be particularly suitable for cytoplasmic targeting.

Antibody types include, but are not limited to, polyclonal, monoclonal, chimeric, single chain, Fab fragments, and an Fab expression library. Various procedures known in the art may be used for the production of polyclonal antibodies to a target protein. For production of the antibody, various host animals can be immunized by injection with the target protein, such host animals include, but are not limited to, rabbits, mice, rats, etc. Various adjuvants can be used to increase the immunological response, depending on the host species, and include, but are not limited to, Freund's (complete and incomplete), mineral gels such as aluminum hydroxide, surface active substances such as lysolecithin, pluronic polyols, polyanions, peptides, oil emulsions, dinitrophenol, and potentially useful human adjuvants such as bacillus Calmette-Guerin (BCG) and corynebacterium parvum.

For preparation of monoclonal antibodies directed towards a target protein, any technique that provides for the production of antibody molecules by continuous cell lines in culture may be used. Such techniques include, but are not restricted to, the hybridoma technique originally developed by Kohler and Milstein (1975, Nature 256: 495-497), the trioma technique, the human B-cell hybridoma technique (Kozbor et al., 1983, Immunology Today 4: 72), and the EBV hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80: 2026-2030), or by transforming human B cells with EBV virus in vitro (Cole et al., 1985, in Monoclonal Antibodies and Cancer Therapy, Alan R. Liss, Inc., pp. 77-96). In fact, according to the invention, techniques developed for the production of “chimeric antibodies” (Morrison et al., 1984, Proc. Natl. Acad. Sci. U.S.A. 81: 6851-6855; Neuberger et al., 1984, Nature 312:604-608; Takeda et al, 1985, Nature 314: 452-454) by splicing the genes from a mouse antibody molecule specific for the target protein together with genes from a human antibody molecule of appropriate biological activity can be used; such antibodies are within the scope of this invention.

Additionally, where monoclonal antibodies are advantageous, they can be alternatively selected from large antibody libraries using the techniques of phage display (Marks et al, 1992, J. Biol. Chem. 267:16007-16010). Using this technique, libraries of up to 10¹² different antibodies have been expressed on the surface of fd filamentous phage, creating a “single pot” in vitro immune system of antibodies available for the selection of monoclonal antibodies (Griffiths et al., 1994, EMBO J. 13:3245-3260). Selection of antibodies from such libraries can be done by techniques known in the art, including contacting the phage to immobilized target protein, selecting and cloning phage bound to the target, and subcloning the sequences encoding the antibody variable regions into an appropriate vector expressing a desired antibody format.

According to the invention, techniques described for the production of single chain antibodies (U.S. Pat. No. 4,946,778) can be adapted to produce single chain antibodies specific to the target protein. An additional embodiment of the invention utilizes the techniques described for the construction of Fab expression libraries (Huse et al., 1989, Science 246: 1275-1281) to allow rapid and easy identification of monoclonal Fab fragments with the desired specificity for the target protein.

Antibody fragments that contain the idiotypes of the target protein can be generated by techniques known in the art. For example, such fragments include, but are not limited to: the F(ab′)₂ fragment which can be produced by pepsin digestion of the antibody molecule; the Fab′ fragments that can be generated by reducing the disulfide bridges of the F(ab′)₂ fragment, the Fab fragments that can be generated by treating the antibody molecule with papain and a reducing agent, and Fv fragments.

In the production of antibodies, screening for the desired antibody can be accomplished by techniques known in the art, e.g., ELISA (enzyme-linked immunosorbent assay). To select antibodies specific to a target protein, one may assay generated hybridomas or a phage display antibody library for an antibody that binds to the target protein.

5.5.5. Methods of Modifying Protein Activities

Methods of directly modifying protein activities include, inter alia, dominant negative mutations, specific drugs (used in the sense of this application) or chemical moieties generally, and also the use of antibodies, as previously discussed.

Dominant negative mutations are mutations to endogenous genes or mutant exogenous genes that when expressed in a cell disrupt the activity of a targeted protein species. Depending on the structure and activity of the targeted protein, general rules exist that guide the selection of an appropriate strategy for constructing dominant negative mutations that disrupt activity of that target (Hershkowitz, 1987, Nature 329:219-222). In the case of active monomeric forms, over expression of an inactive form can cause competition for natural substrates or ligands sufficient to significantly reduce net activity of the target protein. Such over expression can be achieved by, for example, associating a promoter, preferably a controllable or inducible promoter, of increased activity with the mutant gene. Alternatively, changes to active site residues can be made so that a virtually irreversible association occurs with the target ligand. Such can be achieved with certain tyrosine kinases by careful replacement of active site serine residues (Perlmutter et al., 1996, Current Opinion in Immunology 8:285-290).

In the case of active multimeric forms, several strategies can guide selection of a dominant negative mutant. Multimeric activity can be controllably decreased by expression of genes coding exogenous protein fragments that bind to multimeric association domains and prevent multimer formation. Alternatively, controllable over expression of an inactive protein unit of a particular type can sequester wild-type active units in inactive multimers, and thereby decrease multimeric activity (Nocka et al., 1990, EMBO J. 9:1805-1813). For example, in the case of dimeric DNA binding proteins, the DNA binding domain can be deleted from the DNA binding unit, or the activation domain deleted from the activation unit. Also, in this case, the DNA binding domain unit can be expressed without the domain causing association with the activation unit. Thereby, DNA binding sites are tied up without any possible activation of expression. In the case where a particular type of unit normally undergoes a conformational change during activity, expression of a rigid unit can inactivate resultant complexes. For a further example, proteins involved in cellular mechanisms, such as cellular motility, the mitotic process, cellular architecture, and so forth, are typically composed of associations of many subunits of a few types. These structures are often highly sensitive to disruption by inclusion of a few monomeric units with structural defects. Such mutant monomers disrupt the relevant protein activities and can be controllably expressed in a cell.

In addition to dominant negative mutations, mutant target proteins that are sensitive to temperature (or other exogenous factors) can be found by mutagenesis and screening procedures that are well-known in the art.

Also, one of skill in the art will appreciate that expression of antibodies binding and inhibiting a target protein can be employed as another dominant negative strategy.

5.5.6. Drugs of Specific Known Action

Finally, activities of certain target proteins can be controllably altered by exposure to exogenous drugs or ligands. In a preferable case, a drug is known that interacts with only one target protein in the cell and alters the activity of only that one target protein. Graded exposure of a cell to varying amounts of that drug thereby causes graded perturbations of pathways originating at that protein. The alteration can be either a decrease or an increase of activity. Less preferably, a drug is known and used that alters the activity of only a few (e.g., 2-5) target proteins with separate, distinguishable, and non-overlapping effects. Graded exposure to such a drug causes graded perturbations to the several pathways originating at the target proteins.

5.6. Applications of The Invention

The methods and compositions of the present invention are particularly useful for high throughput assays for screening large numbers of cellular constituents, particularly large numbers of genes or gene products, and determining or characterizing their respective biological functions and/or activities. Specifically, using the methods and compositions of the present invention a user can readily determine whether two cellular constituents are functionally related by comparing perturbation responses for the two cellular constituents according to the methods described in Section 5.2 above. If the perturbation responses for the two cellular constituents are correlated (as determined, e.g., according to Equation 4, section 5.2.3) then the two cellular constituents are identified as likely to be functionally related.

The methods and compositions of the invention are useful, not only for identifying cellular constituents from the same species of organism that are likely to be functionally related, but are equally well suited for identifying cellular constituents from different species of organisms that are likely to be functionally related. For example, in one preferred embodiment the methods and compositions of the present invention can be used to identify genes in two or more different species of organism that are likely to have the same biological function in their respective species of organisms. As an example and not by way of limitation, the methods and compositions of the invention can be used to compare the cellular function of a first gene (referred to herein as gene “a”) in a first species of organism (e.g., organism “X”) to the cellular function of a plurality of different genes (e.g., genes b, c, d,e,f, and g) in a second organism (referred to herein as organism “Y”). As those skilled in the art will readily appreciate, in many instances each of the genes b-g from organism Y can have a high sequence similarity (e.g., a high percentage of sequence identity or sequence homology) to the gene a from organism X. However, in most instances at least some of the genes b-g will have cellular functions in organism Y that are different from, and possible even unrelated to, the cellular function of gene a in organism X despite a high sequence similarity.

Using the compositions and methods of the present invention, however, one skilled in the art can readily determine which of the genes b-g in organism Y, if any, are likely to have the same function as gene a in organism X. In particular, using the methods and compositions of the invention, the skilled artisan can readily compare responses for each of the genes a through g to a common perturbation or more preferably, to a common perturbation set or to a common perturbation subset. For example, using Equation 4, section 5.2.3, one skilled in the art can readily determine the correlation of the response profiles for genes a and b (i.e., ρ_{ab}), for genes a and c (i.e., ρ_{ac}),) for genes a and d (i.e., ρ_{ad}) etc. The genes whose response profile have the highest correlation to the response profile for gene a, and most preferably, the gene whose response profile has the highest correlation to the response profile for gene a, are then identified as having a biological function or activity in organism Y that is likely to be identical to the biological function or activity of gene a in organism X. In a preferred embodiment, a functional test is performed in order to determine if the gene in organism Y and gene a in organism X are orthologs, i.e., are genes from different species of organism that have the same biological function in both organism. Such functional tests include, but are not limited to, in vitro complementation analyses or gene complementation studies.

In another exemplary, but also nonlimiting embodiment of the present invention, the methods of the invention can be used in combination with information of sequence similarity. For example, many genes and gene products have multiple homologs, i.e., other genes or gene products of the same organism or different organisms with high sequence similarity. For example, at least four homologs of the coronin protein, which are referred to as coronin-1, coronin-2, coronin-3 and coronin-4, are known to exists in mouse and in human (see, e.g., Okumura et al., 1998, DNA and Cell Biology 17:779-787).

In certain embodiments therefore the methods of the invention can identify genes (e.g., genes “a,” “b,” “c” and “d”) in a first organism, referred to herein as organism X, and a plurality of genes (e.g., genes “α,” “β,” “γ,” “δ”) from a second organism, referred to herein as organism Y which are likely to be functionally related. That is to say, using the methods and compositions of the present invention, a user can identify a plurality of genes (e.g., a, b, c, d, α, β, γ, and δ) from two or more different species of organisms whose response profiles are correlated and which are therefore co-varied. In such embodiments, a user may also use other functional test information to identify which pairs of genes in the two organisms X and Y are, in fact, orthologs. Specifically, those genes or gene products that are determined both to be co-varied and to complement each other in in vitro complementarity experiments are identified as orthologous genes or gene products. In such embodiments, the perturbations of the perturbation set can include, not only drug exposure or target gene mutations that are listed in Section 5.2, above, but also expression of a gene or gene product of interest in a particular cell type of an organism (e.g., expression in hematopoietic cells).

In yet another exemplary and non-limiting embodiment of the invention, the methods and compositions of the invention can also be used to compare genes or gene products from more than two different organisms. Indeed, such comparisons will often be preferred since they can be used to confirm the identification of functional orthologs made by comparing coregulation of genes or gene products between two different organisms. Considering as an example, and not by way of limitation, the comparison of genes from three different species of organism (e.g., organism X, Y and Z), the methods of the invention can be used to identify genes (e.g., x and y) from the first two organisms (X and Y. respectively) that are coregulated. Next, the methods of the invention can be used to identify a gene z from the third organism Z that is coregulated with gene x from organism X. The methods of the invention can then be used to compare the perturbation response profile of the genes y and z to determine whether y and z are, in fact, coregulated. If y and z are determined to be coregulated, the coregulation of the three genes x, y and z is verified and the genes x, y and z are all identified as orthologs.

### 6. EXAMPLES

The following examples are presented by way of illustration of the previously described invention and is not limiting of that description. In particular, the examples presented herein describes the exemplary cross-correlation of a plurality of yeast gene expression profiles from a first strain of yeast to certain mRNA transcription profiles from a second, different strain of yeast. The two strains of yeast used in the following example are: yeast strain ABY11 Mata leu2Δ1 ma3-52 (Dimster-Denk et al., 1999, J. Lipid Res. 40:850-860) used for GRM analysis and strain BY4743 Mata/αhis3Δ/his3Δleu2Δ/leu2Δma3Δ/ma3Δ+/met15Δ+/lys2Δ(Brachmann et al., 1998, Yeast 14:115-32) used for transcript profile analysis.

6.1. Identification of an Informative Subset of Perturbation Conditions

Genome-wide expression profiles were obtained for 1490 different perturbation conditions of the yeast S. cerevisiae using a Genome Reporter Matrix (“GRM”), as described in Dimster-Denk et al., 1999, J. Lipid Res. 40:850-869. The perturbations included, but were not limited to, treatment of the cells with different chemical compounds (including vanillin, ethidium bromide, fluorouracil, tetracycline, methotrexate, pentenoic acid, azoxystrobin, prochloraz, sulfacetimide, sulfamethoxazole, sulfisoxazole, sulfanilamide and asulam to name a few) at various concentrations and targeted mutations to a number of different genes (including pet117, qcr2, fks1, phd1 and sod1, to name a few).

The GRM assay provides, for each perturbation, measurements of gene expression ratios of each gene of the S. cerevisiae genome normalized to a “reference state.” Typically, however, only a small fraction of the genes in the full genome responded to any particular perturbation with a change in expression levels that were significantly above the measurement noise level (i.e., with changes in expression levels that were statistically significant). Thus, as a first step towards identifying a reduced perturbation set, 1330 genes were selected that were significantly up-regulated or down-regulated in response to the different perturbations.

The response profiles for the 1330 selected genes are illustrated graphically in FIG. 3. Specifically, each column of the plot in FIG. 3 represents the response of a particular S. cerevisiae gene to each of the 1490 different perturbations (vertical axis). To facilitate visualization of the different types of responses, the different profiles were clustered according to a two-dimensional hierarchical agglomerative clustering method using the hclust algorithm (MathSoft, Seattle, Wash.) and employing the distance metric and correlation coefficient of Equations 1 and 2, respectively, below. The different genes and perturbation experiments were then reordered and displayed in FIG. 3 according to their clustering similarity. The resulting cluster trees for the genes and perturbation experiments are shown on the top and on the left hand side of FIG. 3, respectively.

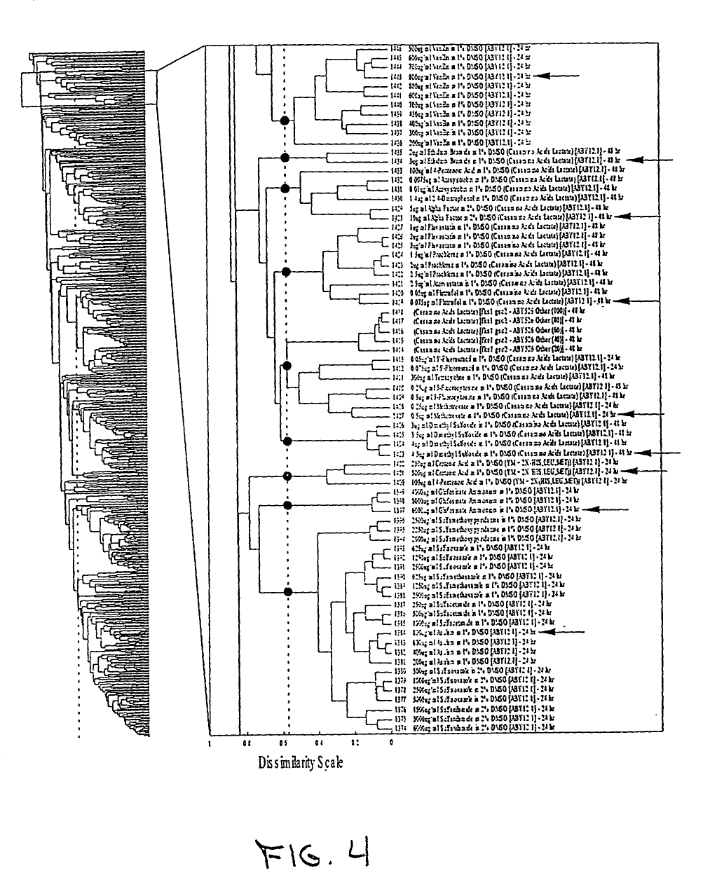

To reduce the perturbation set, a cut-off distance of Dᵢⱼ=0.57 was used to group the 1490 different perturbation conditions into 106 clusters. The hierarchical cluster tree is shown in FIG. 4 (left hand side) with a dashed line indicating the selected cut-off distance of Dᵢⱼ=0.57. An expanded region of the cluster tree is also shown in FIG. 4 (right hand side) to illustrate the selection of representative profiles (indicated by arrows) from nine exemplary clusters (indicated by solid dots). The particular response profile from each cluster which had the largest value of Sᵢ (Equation 3, below) was selected as the representative profile for that cluster.

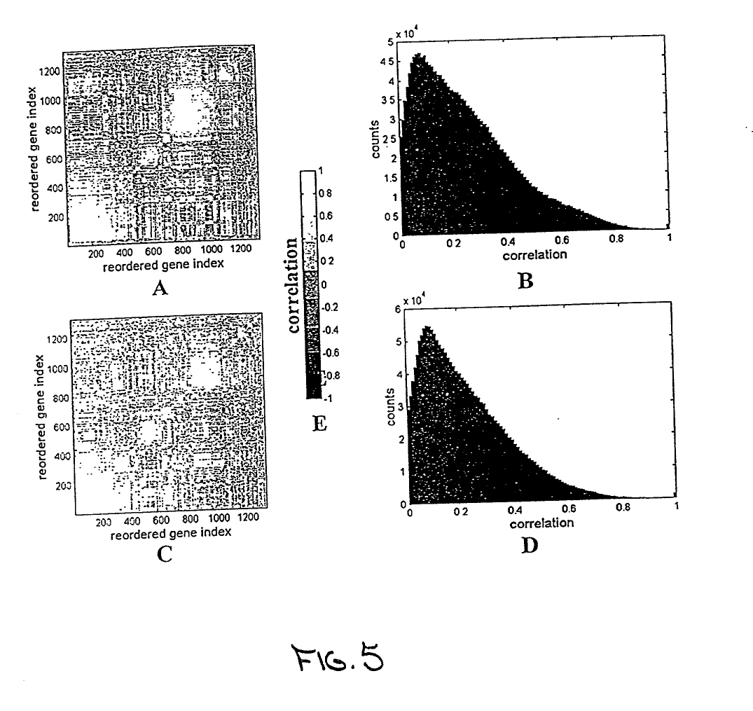

The gene-gene correlations derived from this reduced perturbation subset are similar to, and therefore representative of, the different correlations derived from the entire perturbation set, as demonstrated in FIGS. 5A-5D. In particular, FIG. 5A shows a plot of the gene-gene correlations (determined using Equation 4, section 5.2.3) among the 1330 significant genes based on the GRM profiles under the 1490 perturbation conditions of the fill perturbation set. A plot of the distribution of these correlation values is also shown, in FIG. 5B. The gene-gene correlations among only the 106 selected perturbation conditions of the reduced perturbation subset were also calculated and are plotted in FIG. 5C, along with the distribution of correlation values obtained for this subset (FIG. 5D). Visual comparison of these two correlation plots (i.e., FIGS. 5A and 5C) and their distributions (i.e., FIGS. 5B and 5D) confirms that the gene-gene co-regulations derived from the reduced perturbation subset are similar to, and therefore representative of, the gene-gene co-regulations derived from the full perturbation set.

6.2. Cross-Correlation of Perturbation Responses In Different Strains of S. Cerevisiae

As an exemplary illustration of the methods of the invention, S. cerevisiae expression data from genome reporter matrix (“GRM”) experiments was compared to genome transcript matrix (“GTM”) data. The GRM assay is described in Dimster-Denk et al., 1999, J. Lipid Res. 40:850-860. Briefly, the GRM assay is a method for obtaining an expression profile in which a collection of strains of S. Cerevisiae, each containing a reporter gene fused to a different protein-coding gene, is subjected to a perturbation. The reporter gene response in each strain is measured and is collectively referred to as the “expression profile” that is responsive to the perturbation. Because each reporter gene fusion in each strain of S. Cerevisiae includes the promoter region as well as the first few codons of the individual open reading frames (“ORFs”) associated with the reporter gene, the GRM assay provides a readout of both the transcriptional and translational components of gene expression. Thus, the GRM assay provides a method for obtaining a profile that is a combination of the transcript and protein abundance. The GTM assay likewise is a method to obtain an expression profile but uses DNA microarrays in a manner that is described in section 5.4.2.

The two strains of yeast used in this example are: yeast strain ABY11 Mata leu2Δ1 ma3-52 (Dimster-Denk et al., 1999, J. Lipid Res. 40:850-860), which is used for GRM analysis (experiments 1-16), and yeast strain BY4743 Mata/αhis3Δ/his3Δ/leu2Δma3Δ/ma3Δ+/met15Δ+/lys2Δ(Brachmann et al., 1998, Yeast 14:115-32), which is used for GTM analysis (experiments 17-32). Drug exposures in experiments 17-32 were for approximately six hours.

In this example, sixteen perturbation conditions were profiled in the GRM assay and sixteen similar perturbation conditions were profiled in the GTM transcript assay. Thus, a reduced perturbation set consisting of sixteen conditions for the GRM assay and sixteen conditions for the GTM assay were used to identify functional homologs among the two strains of S. cerevisiae. The perturbations used in the two assays are listed below in Table 1. In Table 1, GTM experiments 1-16 respectively correspond to GRM experiments 17-32. For example, experiment 1 (GTM) corresponds to experiment 17 (GRM) (exposure to clotrimazole), experiment 2 (GTM) corresponds to experiment 18 (GRM) (exposure to miconazole) and so forth. In total, 335 genes responded significantly (P<0.05) to the perturbations.

TABLE 1

| Exps # | Type | Perturbation |
|---|---|---|
| 1 | GTM | Exposure of cells to 0.12 μg/ml clotrimazole in a one |
| | | percent DMSO solution for 24 hours. |
| 2 | GTM | Exposure of cells to 0.03 μg/ml miconazole in a one |
| | | percent DMSO solution for 24 hours. |
| 3 | GTM | Exposure of cells to 1.25 μg/ml ketoconazole in a one |
| | | percent DMSO solution for 24 hours. |
| 4 | GTM | Effect of reduced expression of ERG 11 |
| 5 | GTM | Exposure of cells to 0.25 μg/ml 5-fluorouracil in a one |
| | | percent DMSO solution for 24 hours. |
| 6 | GTM | Exposure of cells to 100 μg/ml methotrexate in a one |
| | | percent DMSO solution for 24 hours. |
| 7 | GTM | Exposure of cells to 0.35 μg/ml haloprogin in a one |
| | | percent DMSO solution for 24 hours. |
| 8 | GTM | Exposure of cells to 5500 μg/ml hydroxyurea in a one |
| | | percent DMSO solution for 24 hours. |
| 9 | GTM | Exposure of cells to 60 μg/ml of undecylenic acid in a |
| | | one percent DMSO solution for 24 hours. |
| 10 | GTM | Exposure of cells to 100 μg/ml cyclosporin A in a two |
| | | percent DMSO solution for 24 hours. |
| 11 | GTM | Exposure of cells to 200 μg/ml doxycycline in a one |
| | | percent DMSO solution for 24 hours. |
| 12 | GTM | Effect of reduced expression of ERG 13 |
| 13 | GTM | Exposure of cells to 10 μg/ml atorvastatin in a one |
| | | percent DMSO solution for 24 hours. |
| 14 | GTM | Exposure of cells to 6 μg/ml fluvastatin in a one percent |
| | | DMSO for 24 hours. |
| 15 | GTM | Exposure of cells to 20 μg/ml simvastatin in a one |
| | | percent DMSO solution for 24 hours. |
| 16 | GTM | Exposure of cells to 5 μg/ml lovastatin in one percent |
| | | DMSO for 24 hours. |
| 17 | GRM | Exposure of BY4743 cells to 1 μg/ml clotrimazole, |
| | | compared to mock treated cells. |
| 18 | GRM | Exposure of BY4743 cells to 0.1 μg/ml miconazole |
| | | compared to mock treated cells. |
| 19 | GRM | Exposure of BY4743 cells to 12 μg/ml ketoconazole, |
| | | compared to mock treated cells. |
| 20 | GRM | Effect of reduced expression of ERG11, compared to |
| | | wild-type cells by replacing the chromosomal copy of the |
| | | ERG11 gene with an ERG11 gene under control of the |
| | | tet promoter (denoted the tet-ERG11 strain); exposure of |
| | | the tet-ERG11 strain to 1 μg/ml doxycyline. |
| 21 | GRM | Exposure of BY4743 cells to 50 μM 5-fluorouracil, |
| | | compared to mock treated cells. |
| 22 | GRM | Exposure of BY4743 cells to 200 μM methotrexate, |
| | | compared to mock treated cells. |
| 23 | GRM | Exposure of BY4743 cells to 0.04 μg/ml haloprogin, |
| | | compared to mock treated cells. |
| 24 | GRM | Exposure of BY4743 cells to 50 mM hydroxyurea, |
| | | compared to mock treated cells. |
| 25 | GRM | Exposure of BY4743 cells to 4 μg/ml undecylenic acid, |
| | | compared to mock treated cells. |
| 26 | GRM | Exposure of BY4743 cells to 50 μg/ml cyclosporin A, |
| | | compared to mock treated cells. |
| 27 | GRM | Exposure of BY4743 cells to 100 μg/ml doxycyline, |
| | | compared to mock treated cells. |
| 28 | GRM | Effect of reduced expression of HMG2, compared to wild |
| | | type cells. The chromosomal copy of the HMG2 gene |
| | | was replaced with a HMG2 gene under control of the |
| | | tet promoter (denoted tet-HMG2). The tet-HMG2 strain |
| | | was treated with 300 μg/ml doxycyline, which |
| | | represses transcription form the tet promoter, and |
| | | compared to wild-type cells treated with |
| | | 300 μg/ml doxycyline. |
| 29 | GRM | Exposure of BY4743 cells to 31.62 μg/ml atorvastatin, |
| | | compared to mock treated cells. |
| 30 | GRM | Exposure of BY4743 cells to 31.62 μg/ml fluvastatin, |
| | | compared to mock treated cells. |
| 31 | GRM | Exposure of BY4743 cells to 31.62 μg/ml simvastatin, |
| | | compared to mock treated cells. |
| 32 | GRM | Exposure of BY4743 cells to 31.62 μg/ml lovastatin, |
| | | compared to mock treated cells. |

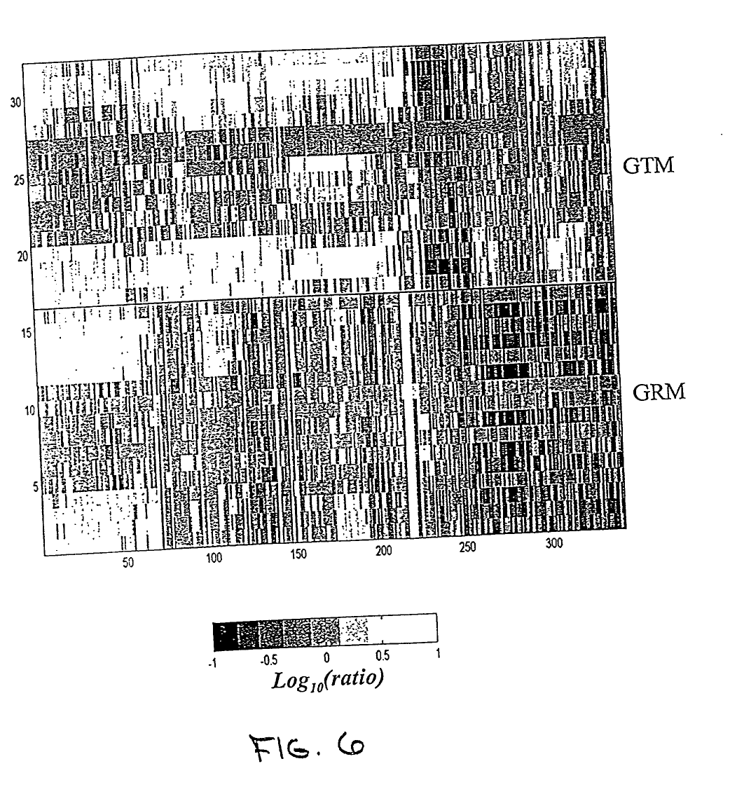

The data from the GTM assay and the GRM assays are depicted in the top and bottom halves, respectively, of the plot in FIG. 6. Thus, FIG. 6 is the logarithmic plot of the expression ratios for 335 genes (horizontal axis) under sixteen corresponding perturbation conditions that were measured in each of the GRM and GTM assays. To analyze the experiments listed in Table 1, a correlation coefficient for the expression ratio between the GTM and GRM assays of each of the 335 genes was computed using Equation 4 (see section 5.2.3). The 35 highest correlations are summarized in descending order in Table 2 along with a brief description of the “substance,” a systematic name given to all predicted genes (which may not be real genes at all, or which may not have a known function), the “gene,” which describes the experimentally-derived function, and a description of the protein encoded by it. Thus Table 2 lists the counterpart genes which co-vary most similarly in the GRM and GTM experiments. The large correlation values (ρ≧0.8) listed in Table 2 are indicative of functional homology between corresponding genes in ABY11 and BY4743.

TABLE 2

| Index | Correlation | Substance | Gene | Protein Description |
|---|---|---|---|---|
| 1 | 0.9606 | YFL020C | PAU5 | strong similarity to members of the |
| | | | | Srp1p/Tip1p family |
| 2 | 0.9500 | YPL272C | | hypothetical protein |
| 3 | 0.9360 | YBR301W | | strong similarity to members of the |
| | | | | Srp1p/Tip1p family |
| 4 | 0.9335 | YOR237W | HES1 | involved in ergosterol biosynthesis |
| 5 | 0.9220 | YDR213W | | regulatory protein involved in control |
| | | | | of sterol uptake |
| 6 | 0.9147 | YNR076W | PAU6 | strong similarity to members of the |
| | | | | Tir1p/Tip1p family |
| 7 | 0.9134 | YEL049W | PAU2 | strong similarity to members of the |
| | | | | Srp1p/Tip1p family |
| 8 | 0.9067 | YLL012W | | similarity to triacylglycerol lipases |
| 9 | 0.9044 | YLR461W | PAU4 | strong similarity to members of the |
| | | | | Tir1p/Tip1p family |
| 10 | 0.9042 | YKL224C | | strong similarity to members of the |
| | | | | Srp1p/Tip1p family |
| 11 | 0.8951 | YPL254W | HFI1/ | transcriptional coactivator |
| | | | ADA1/ | |
| | | | SUP110 | |
| 12 | 0.8905 | YMR220W | ERG8 | phosphomevalonate kinase |
| 13 | 0.8817 | YHR209W | | putative methyltransferase |
| 14 | 0.8794 | YMR325W | | strong similarity to members of the |
| | | | | Srp1p/Tip1p family |
| 15 | 0.8783 | YOR034C | AKR2 | involved in constitutive endocytosis of |
| | | | | Ste3p |
| 16 | 0.8698 | YHR030C | SLT2/ | ser/thr protein kinase of MAP kinase |
| | | | BYC2/ | family |
| | | | MPK1/ | |
| | | | SLK2 | |
| 17 | 0.8631 | YGR294W | | strong similarity to members of the |
| | | | | Srp1p/Tip1p family |
| 18 | 0.8561 | YPR167C | MET16 | 3′-phosphoadenuylylsulfate reductase |
| 19 | 0.8535 | YLR431C | | weak similarity to rabbit trichohyalin |
| 20 | 0.8448 | YMR316W | | similarity to YOR385w and |
| | | | | YNL165W |
| 21 | 0.8428 | YKR091W | | similarity to YOR083w |
| 22 | 0.8397 | YJR150C | DAN1 | conditions |
| 23 | 0.8314 | YOR134W | BAG7 | structural homolog of Sac7p |
| 24 | 0.8235 | YOR009W | | similarity to Tir1p and Tir2p |
| 25 | 0.8213 | YJR130C | | similarity to O-succinylhomoserine |
| | | | | (thiol)-lyase |
| 26 | 0.8195 | YCR048W | ARE1/ | acyl-CoA sterol acyltransferase |
| | | | SAT2 | |
| 27 | 0.8191 | YKL072W | STB6 | SIN3 binding protein |
| 28 | 0.8175 | YPL088W | | similarity to aryl-alcohol |
| | | | | dehydrogenases |
| 29 | 0.8159 | YGL261C | | strong similarity to members of the |
| | | | | Srp1/Tip1 family |
| 30 | 0.8155 | YPR198W | SGE1/ | drug resistance protein |
| | | | NOR1 | |
| 31 | 0.8129 | YMR317W | | similarity to mucins, glucan |
| | | | | 1,4-alpha-glucosidase and |
| | | | | exo-alpha-sialidase |
| 32 | 0.8122 | YOR011W | | strong similarity to ATP-dependent |
| | | | | permeases |
| 33 | 0.8102 | YPR015C | | similarity to transcription factors |
| 34 | 0.8080 | YJL131C | | weak similarity to nonepidermal |
| | | | | Xenopus keratin, type I |
| 35 | 0.8024 | YHL046C | | strong similarity to members of the |
| | | | | Srp1p/Tip1p family |

In addition to intra-species comparisons, the methods and compositions described herein are applicable to the comparison of gene-gene correlations between different species. For example, the methods described herein, including the particular exemplary methods described in this example, can be readily used to evaluate cross-correlation between genes, e.g., of S. cerevisiae and C. albicans; of S. pombe and C. albicans; and/or between all three organisms (e.g., among S. cerevisiae, S. pombe and C albicans). In such instances, the format Table 2 would not necessarily include a generic protein description. Rather, when an inter-species comparison is made (i.e. a comparison of profiles between two different species), one column in the table tracks “substance-species A,” a second column tracks “substances-species B,” and a third column tracks the correlation between the two substances, where “substance” is a systematic name given to all predicted genes, which may not be real genes at all, or which may not have a known function. It is expected that some inter-species comparisons will co-vary so closely that the correlation between two different genes could be greater than 0.85, and thus besides the “actual” functional homolog between the two species (i.e., the actual corresponding gene in the two species), genes that are “functional candidates” between the two strains could be identified, where a functional candidate is defined in one embodiment of the invention as having a correlation greater than 0.85 in the inter-species comparison. In an exemplary embodiment, a table lists genes from the GRM strain and the table includes a column that identifies “functional homolog candidates” from the GTM strain. In this way, for example, a substance such as YFL020c from the GRM strain is listed and genes in the GTM strain with correlation values greater than 0.85 are identified. Genes having a correlation of 0.85 to YFL020c are likely to be YFL020c functional homology candidates in the GTM strain.

### 7. REFERENCES CITED

All publications, patents and patent applications cited herein are incorporated herein by reference in their entirety and for all purposes to the same extent as if each individual publication or patent or patent application was specifically and individually indicated to be incorporated by reference in its entirety for all purposes.

Many different modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed is:

1. A method for identifying a functional homolog of a cellular constituent, said method comprising comparing a response profile for a cellular constituent of a first cell or organism to a response profile for a cellular constituent of a second cell or organism to determine whether said cellular constituent is coregulated, wherein the determination that said cellular constituent is coregulated identifies said cellular constituent of said second cell or organism as said functional homolog of said cellular constituent of said first cell or organism.

2. The method of claim 1 wherein said step of comparing comprises determining a correlation of said response profile for said cellular constituent of said first cell or organism to said response profile for said cellular constituent of said second cell or organism is determined.

3. The method of claim 2, wherein said correlation is determined in accordance with the equation:
$${ρ}_{xy} = \frac{{\sum }_{i} {x}_{i} {\sum }_{i} {y}_{i}}{{( {\sum }_{i} {x}_{i}^{2} {\sum }_{i} {y}_{i}^{2} )}^{1 / 2}}$$
where
ρ_{xy} is said correlation;
xᵢ denotes an expression level, an abundance, an activity level, or an amount of modification of a gene product corresponding to said cellular constituent of said first cell or organism;
yᵢ denotes an expression level, an abundance, an activity level, or an amount of modification of a gene product corresponding to said cellular constituent of said second cell or organism; and
i is a perturbation in a plurality of perturbations used to derives said response profile for said cellular constituent of said first cell or organism and said response profile for said cellular constituent of said second cell or organism.

4. The method of claim 2, wherein said correlation is determined in accordance with the equation:
$${ρ}_{xy} = \frac{{\sum }_{iX} {x}_{iX} {\sum }_{iY} {y}_{iY}}{{( {\sum }_{iX} {x}_{iX}^{2} {\sum }_{iY} {y}_{iY}^{2} )}^{1 / 2}}$$
where ρ_{xy} is said correlation;
iX is a perturbation applied to said first cell or organism;
iY is a perturbation applied to said second cell or organism;
x_{iX} denotes an expression level, an abundance, an activity level, or an amount of modification of a gene product corresponding to said cellular constituent of said first cell or organism; and
y_{iY} denotes an expression level, an abundance, an activity level, or an amount of modification of a gene product corresponding to said cellular constituent of said second cell or organism; and

5. The method of claim 2 wherein said cellular constituent of said second cell or organism is identified as said functional homolog of said cellular constituent of said first cell or organism if the correlation of said response profile for said cellular constituent of said first cell or organism to said response profile for said cellular constituent of said second cell or organism is at least 50%.

6. The method of claim 5 wherein said cellular constituent of said second cell or organism is identified as said functional homolog of said cellular constituent of said first cell or organism if the correlation of said response profile for said cellular constituent of said first cell or organism to said response profile for said cellular constituent of said second cell or organism is at least 75%.

7. The method of claim 6 wherein said cellular constituent of said second cell or organism is identified as said functional homolog of said cellular constituent of said first cell or organism if the correlation of said response profile for said cellular constituent of said first cell or organism to said response profile for said cellular constituent of said second cell or organism is at least 80%.

8. The method of claim 7 wherein said cellular constituent of said second cell or organism is identified as a functional homolog of said cellular constituent of said first cell or organism if the correlation of said response profile for said cellular constituent of said first cell or organism to said response profile for said cellular constituent of said second cell or organism is at least 85%.

9. The method of claim 8 wherein said cellular constituent of said second cell or organism is identified as said functional homolog of said cellular constituent of said first cell or organism if the correlation of said response profile for said cellular constituent of said first cell or organism to said response profile for said cellular constituent of said second cell or organism is at least 90%.

10. The method of claim 1 wherein said response profile for said cellular constituent of said first cell or organism comprises differential measurements of changes in said cellular constituent of said first cell or organism in response to a plurality of perturbations to said first cell or organism.

11. The method of claim 1 wherein said response profile for said cellular constituent of said second cell or organism comprises differential measurements of changes in said cellular constituent of said second cell or organism in response to a plurality of perturbations to said first cell or organism.

12. The method of claim 1 wherein
said response profile for said cellular constituent of said first cell or organism comprises differential measurements of changes in said cellular constituent of said first cell or organism in response to a plurality of perturbations to said first cell or organism,
said response profile for said cellular constituent of said second cell or organism comprises differential measurements of changes in said cellular constituent of said second cell or organism in response to a plurality of perturbations to said second cell or organism, and
said plurality of perturbations to said second cell or organism are the same as said plurality of perturbations to said first cell or organism.

13. The method of any one of claims 10-12, wherein said plurality of perturbations comprises at least 50 different perturbations.

14. The method of claim 13, wherein said plurality of perturbations comprises at least 100 different perturbations.

15. The method of claim 14, wherein said plurality of perturbations comprises between 100 and 500 different perturbations.

16. The method of claim 10, wherein a perturbation subset is identified,
said perturbation subset consisting of selected perturbations from said plurality of perturbations to said first cell or organism, and
wherein changes in cellular constituents of said first cell or organism in response to said selected perturbations are maximally informative.

17. The method of claim 16, wherein said perturbation subset comprises at least 50 perturbations.

18. The method of claim 17, wherein said perturbation subset comprises at least 100 perturbations.

19. The method of claim 18, wherein said perturbation subset comprises between 100 and 500 perturbations.

20. The method of claim 16, wherein selected perturbations are selected from said plurality of perturbations to said first cell or organism according to a method comprising:
(a) clustering the perturbations of said plurality of perturbations to said first cell or organism into cluster groups according to similarities between responses of cellular constituents of said first cell or organism to the perturbations of said plurality of perturbations to said first cell or organism; and
(b) selecting a representative perturbation from each of said cluster groups.

21. The method of claim 20 wherein the perturbations of said plurality of perturbations are clustered into at least 50 cluster groups.

22. The method of claim 21 wherein the perturbations of said plurality of perturbations are clustered into at least 100 cluster groups.

23. The method of claim 22, wherein the perturbations of said plurality of perturbations are clustered into between 100 and 500 cluster groups.

24. The method of claim 20, wherein the representative perturbation selected from a particular cluster group is the perturbation of the particular cluster group which produces the most significant changes in said cellular constituents of said first cell or organism.

25. The method of any one of claims 10-12, wherein said plurality of perturbations comprises exposure to one or more drugs.

26. The method of any one of claims 10-12, wherein said plurality of perturbations comprises one or more mutations.

27. The method of any one of claims 10-12, wherein said plurality of perturbations comprises one or more changes in protein activity.

28. The method of any one of claims 10-12, wherein said plurality of perturbations comprises a change in environmental conditions.

29. The method of any one of claims 10-12, wherein said plurality of perturbations comprises exposure to one or more toxins.

30. The method of claim 1, wherein said cellular constituent of said first cell or organism is a gene of said first cell or organism.

31. The method of claim 1, wherein said cellular constituent of said second cell or organism is a gene of said second cell or organism.

32. The method of claim 1, wherein said cellular constituent of said first cell or organism is a gene product of said first cell or organism.

33. The method of claim 32, wherein said gene product is a protein.

34. The method of claim 1, wherein said cellular constituent of said second cell or organism is a gene product of said second cell or organism.

35. The method of claim 34, wherein said gene product is a protein.

36. The method of claim 1, wherein said second cell or organism is different from said first cell or organism.

37. The method of claim 36, wherein:
said first cell or organism is a cell of a first species of organism;
said second cell or organism is a cell of a second species of organism; and
said second species of organism is different from said first species of organism.

38. The method of claim 36, wherein:
said first cell or organism is a first cell type of a first organism;
said second cell or organism is a second cell type of a second organism; and
said second cell type is different from said first cell type.

39. The method of claim 38, wherein said first organism and said second organism are the same organism.

40. The method of claim 38 wherein said first organism and said second organism are the same species of organism.

41. The method of claim 38, wherein said first organism and said second organism are different species of organism.

42. A computer system for identifying a functional homolog of a cellular constituent, said computer system comprising:
a memory to store instructions and data;
a processor to execute the instructions stored in memory; and
the memory storing:
(a) a response profile for a cellular constituent of a first cell or organism;
(b) a response profile for a cellular constituent of a second cell or organism;
(c) instructions for determining a correlation of said response profile for said cellular constituent of said first cell or organism to said response profile for said cellular constituent of said second cell or organism; and
(d) instructions for determining whether said correlation is above a threshold value, wherein said cellular constituent of said second cell or organism is identified as a functional homolog of said cellular constituent of said second cell or organism when said correlation is at least equal to said threshold value.

43. A computer system for identifying a functional homolog of a cellular constituent, said computer system comprising:
a memory to store instructions and data;
a processor to execute the instructions stored in memory; and
the memory storing:
(a) instructions for determining a correlation of a response profile for a cellular constituent of a first cell or organism to a response profile for a cellular constituent of a second cell or organism; and
(b) instructions for determining whether said correlation is above a threshold value, wherein said cellular constituent of said second cell or organism is identified as a functional homolog of said cellular constituent of said second cell or organism when said correlation is at least equal to said threshold value.

44. The computer system of claim 42 or 43, the memory further storing instructions for determining said correlation in accordance with the equation:
$${ρ}_{xy} = \frac{{\sum }_{i} {x}_{i} {\sum }_{i} {y}_{i}}{{( {\sum }_{i} {x}_{i}^{2} {\sum }_{i} {y}_{i}^{2} )}^{1 / 2}}$$
where
ρ_{xy} is said correlation;
xᵢ denotes an expression level, an abundance, an activity level, or an amount of modification of a gene product corresponding to said cellular constituent of said first cell or organism;
yᵢ denotes an expression level, an abundance, an activity level, or an amount of modification of a gene product corresponding to said cellular constituent of said second cell or organism; and
i is a perturbation in a plurality of perturbations used to derives said response profile for said cellular constituent of said first cell or organism and said response profile for said cellular constituent of said second cell or organism.

45. The computer system of claim 42 or 43, the memory further storing instructions for determining said correlation in accordance with the equation:
$${ρ}_{xy} = \frac{{\sum }_{iX} {x}_{iX} {\sum }_{iY} {y}_{iY}}{{( {\sum }_{iX} {x}_{iX}^{2} {\sum }_{iY} {y}_{iY}^{2} )}^{1 / 2}}$$
where ρ_{xy} is said correlation;
iX is a perturbation applied to said first cell or organism;
iY is a perturbation applied to said second cell or organism;
X_{iX} denotes an expression level, an abundance, an activity level, or an amount of modification of a gene product corresponding to said cellular constituent of said first cell or organism; and
Y_{iY} denotes an expression level, an abundance, an activity level, or an amount of modification of a gene product corresponding to said cellular constituent of said second cell or organism; and

46. The computer system of claim 42 or 43, wherein said cellular constituent of said second cell or organism is identified as said functional homolog of said cellular constituent of said second cell or organism if said correlation is at least 50%.

47. The computer system of claim 42 or 43, wherein said cellular constituent of said second cell or organism is identified as said functional homolog of said cellular constituent of said second cell or organism if said correlation is at least 75%.

48. The computer system of claim 42 or 43, wherein said cellular constituent of said second cell or organism is identified as said functional homolog of said cellular constituent of said second cell or organism if said correlation is at least 80%.

49. The computer system of claim 42 or 43, wherein said cellular constituent of said second cell or organism is identified as said functional homolog of said cellular constituent of said second cell or organism if said correlation is at least 85%.

50. The computer system of claim 42 or 43, wherein said cellular constituent of said second cell or organism is identified as said functional homolog of said cellular constituent of said second cell or organism if said correlation is at least 90%.

51. The computer system of claim 42 or 43, the memory further storing instructions for accepting said response profile for said cellular constituent of said first cell or organism or said response profile for said cellular constituent of said second cell or organism from a user.

52. The computer system of claim 42 or 43, the memory further storing instructions for reading said response profile for said cellular constituent of said first cell or organism or said response profile for said cellular constituent of said second cell or organism from a database.

53. The computer system of claim 42 or 43, wherein said response profile for said cellular constituent of said first cell or organism comprises differential measurements of changes in said cellular constituent of said first cell or organism in response to a plurality of perturbations to said first cell or organism.

54. The computer system of claim 42 or 43, wherein said response profile for said cellular constituent of said second cell or organism comprises differential measurements of changes in said cellular constituent of said second cell or organism in response to a plurality of perturbations to said second cell or organism.

55. The computer system of claim 42 or 43 wherein:
said response profile for said cellular constituent of said first cell or organism comprises differential measurements of changes in said cellular constituent of said first cell or organism in response to a plurality of perturbations to said first cell or organism;
said response profile for said cellular constituent of said second cell or organism comprises differential measurements of changes in said cellular constituent of said second cell or organism in response to a plurality of perturbations to said second cell or organism; and
said plurality of perturbations to said second cell or organism is the same as said plurality of perturbations to said first cell or organism.

56. The computer system of claim 53, the memory further storing instructions for identifying a perturbation subset consisting of selected perturbations from said plurality of perturbations to said first cell or organism; wherein a change in a cellular constituent of said first cell or organism in response to said selected perturbations is maximally informative.

57. The computer system of claim 56, the memory further storing instructions for selecting said selected perturbations of said perturbation subset by a method comprising:
(a) clustering the perturbations of said plurality of perturbations to said first cell or organism into cluster groups according to similarities between responses of cellular constituents of said first cell or organism to the perturbations of said plurality of perturbations to said first cell or organism; and
(b) selecting a representative perturbation from each of said cluster groups.

58. The computer system of claim 57, the memory further storing instructions for selecting said representative perturbation from each of said cluster groups by selecting, for each of said cluster groups, a perturbation which produces the most significant changes in said cellular constituents of said first cell or organism.

59. A computer program product for use in conjunction with a computer having a processor and memory connected to the processor,
said computer program product comprising a computer readable storage medium having a computer program mechanism encoded thereon, wherein the computer program mechanism can be loaded into the memory of the computer and cause the processor to execute the steps of:
(a) determining the correlation of a response profile for a cellular constituent of a first cell or organism to a response profile for a cellular constituent of a second cell or organism; and
(b) deciding whether said correlation is above a threshold value, so that said cellular constituent of said second cell or organism is identified as a functional homolog of said cellular constituent of said second cell or organism if said correlation is equal to or greater than said threshold value.

60. The computer program product of claim 59, wherein said computer program mechanism can further cause the processor of the computer to accept one or more response profiles entered into memory by a user.

61. The computer program product of claim 59, wherein said computer program mechanism can further cause the processor of the computer to read one or more response profiles from a database.

62. The computer program product of claim 61, further comprising a database of response profiles for one or more cellular constituents, each said response profile comprising differential measurements of changes in a cellular constituent is response to a plurality of perturbations to a cell or organism.

63. The computer program product of claim 59, wherein said response profile for said cellular constituent of said first cell or organism comprises differential measurements of changes in said cellular constituent of said first cell or organism in response to a plurality of perturbations to said first cell or organism.

64. The computer program product of claim 59, wherein said response profile for said cellular constituent of said second cell or organism comprises differential measurements of changes in said cellular constituent of said second cell or organism in response to a plurality of perturbations to said second cell or organism.

65. The computer program product of claim 59, wherein said computer program mechanism further causes the processor to identify a perturbation subset consisting of selected perturbations from said plurality of perturbations to said first cell or organism, wherein a change in a cellular constituent of said first cell or organism in response to said selected perturbations is maximally informative.

66. The computer program product of claim 65, said computer program mechanism further causing the processor to identify said selected perturbations of said perturbation subset by a method comprising:
(a) clustering the perturbations of said plurality of perturbations to said first cell or organism into cluster groups according to similarities between responses of cellular constituents of said first cell or organism to the perturbations of said plurality of perturbations to said first cell or organism; and
(b) selecting a representative perturbation from each of said cluster groups.

67. The computer program product of claim 66, said computer program mechanism further causing the processor to select said representative perturbation from each of said cluster groups by selecting, for each of said cluster groups, a perturbation that produces the most significant changes in said cellular constituents of said first organism.
